(19) United States
(12) Patent Application Publication
(10) Pub. No.: US 2018/0153978 A1
(43) Pub. Date: Jun. 7, 2018

(54) TUMORS EXPRESSING IgG1 Fc INDUCE ROBUST CD8 T CELL RESPONSES

(71) Applicants: The Board of Regents of the University of Texas System, Austin, TX (US); Yale University, New Haven, CT (US); The United States of America, as represented by the Secretary, Department of Health and Human Serv, Bethesda, MD (US)

(72) Inventors: Chandrashekhar PASARE, Coppell, TX (US); Scott N. FURLAN, Seattle, WA (US); Noah W. PALM, New Haven, CT (US); Arun UNNI, Washington, DC (US)

(73) Assignees: The Board of Regents of the University of Texas System, Austin, TX (US); Yale University, New Haven, CT (US); The United States of America, as represented by the Secretary, Department of Health and Human Serv, Bethesda, MD (US)

(21) Appl. No.: 15/887,456

(22) Filed: Feb. 2, 2018

Related U.S. Application Data
(62) Division of application No. 15/034,113, filed on May 3, 2016, now abandoned.
(60) Provisional application No. 61/900,100, filed on Nov. 5, 2013.

(51) Int. Cl. A61K 39/00; C12N 5/09; A61K 48/00

(52) U.S. Cl. CPC A61K 39/0011; C12N 5/0693; A61K 48/00; A61K 2039/572; A61K 2039/5152; C12N 2510/00; A61K 2039/5156

(57) ABSTRACT

A lymphoma cell line was engineered to express surface IgG1 Fc. These tumor cells were taken up rapidly by DCs, leading to enhanced cross-presentation of tumor-derived antigen to CD8 T cells. IgG1-Fc tumors failed to grow in vivo and prophylactic vaccination in an animal model resulted in rejection of unmanipulated tumor cells. Furthermore, IgG1-Fc tumor cells were able to slow the growth of an unmanipulated primary tumor when used as a therapeutic tumor vaccine. This demonstrates that engagement of Fc receptors by tumors expressing the Fc region of IgG1 can induce efficient and protective anti-tumor CD8+ T cell responses without prior knowledge of tumor-specific antigen.

The present application is a divisional of U.S. application Ser. No. 15/034,113, filed May 3, 2016, which is a national phase application under 35 U.S.C. § 371 of International Application No. PCT/US2014/064096, filed Nov. 5, 2014, which claims benefit of priority to U.S. Provisional Application Ser. No. 61/900,100, filed Nov. 5, 2013, the entire contents of each of which are hereby incorporated by reference.

### BACKGROUND

### 1. Field of the Invention

The present disclosure relates in general to the field of medicine, immunology and oncology, and more particularly, the preparation and use of engineered tumor cells to treat cancer.

### 2. Related Art

Current anti-cancer treatments are comprised of various combinations of surgery, radiotherapy, chemotherapy and molecularly targeted therapies. The efficacy of many of these therapies is limited by their toxicity and inability to eliminate all tumor cells (Wagle et al., 2011). Despite extensive progress in modifying tumor-specific T cells (Rosenberg et al., 2008) and advances in dendritic cell therapy (Palucka et al., 2012), cancer immunotherapy is still viewed as a complex and confounding therapeutic. This comes as no surprise, considering the number of mechanisms by which tumors bypass immune checkpoints (Pardol, 2012) and thus immune-mediated clearance.

Antigen-presenting dendritic cells (DCs) form a critical link between the innate and adaptive immune systems. When naïve DCs encounter pathogens, they recognize microbial products leading to upregulation of surface major histocompatibility complex (MHC) molecules, costimulatory molecules and production of inflammatory cytokines, such as IL-6, IL-12 and type I interferons (Iwasaki and Medzhitov, 2010). Mature DCs then migrate to draining lymph nodes where they present antigen and prime CD4 and CD8 T cells (Iwasaki and Medzhitov, 2010). A number of current cancer immunotherapy strategies rely on differentiating CD34+ peripheral blood stem cells or monocytes into DCs ex vivo, pulsing them with tumor antigen and infusing them into patients with the hope of inducing effective CD4 and CD8 T cell responses against tumors (Palucka et al., 2012). This approach has had measurable clinical success (Kantoff et al., 2010), but a number of factors may limit its efficacy. First, the many subsets of DCs in vivo differ broadly in their capacity to activate T cells (Joffre et al., 2012). Second, ex vivo manipulated DCs display altered patterns of expression of adhesion molecules and chemokine receptors, which may affect their ability to efficiently migrate to lymphoid organs and prime naïve T cells against the tumor antigen (Topalian et al., 2011). Third, injected DCs have a short half-life in vivo and, without persistent antigen presentation, the magnitude of activation and differentiation of T cells could be variable depending on the quality of the injected DCs (Schuler et al., 2003; Bousso et al., 2003). Finally, and perhaps most importantly, infusion of tumor-antigen loaded DCs into patients requires prior knowledge of which tumor-specific antigens or peptides induce effective anti-tumor immunity (Schuler et al., 2003).

T cell responses to infection are driven largely by pattern recognition receptor (PRR)-mediated detection of conserved pathogen associated molecular patterns (PAMPs) by DCs (Iwasaki and Medzhitov, 2010). As tumors are autologous, they inherently lack many of the patterns that would elicit a productive immune response to infection/microbial non-self (Janeway, 1989). However, a number of phagocytic and endocytic receptors, including Fc receptors, scavenger receptors and mannose receptors, could potentially be exploited to target tumors to dendritic cells (Palucka et al., 2012; Flinsenberg et al., 2012; Cruz et al., 2011). Such targeting is likely to enhance uptake of tumor cells by DCs and lead to the presentation of tumor-derived antigens on MHC molecules (Steinman, 2012). Concomitant activation of PRRs could then provide additional signals aiding induction of optimal effector responses against tumor cells (Cruz et al., 2011).

Four classes of IgG Fc receptors (FcγR) are expressed widely on cells of both the myeloid and lymphoid lineages, and impart effector functions to IgG subclasses (Nimmerjahn and Ravetch, 2008). Of these, FcγRIIB and FcγRIII predominantly bind to IgG1, the dominant IgG isotype found in mouse serum (Nimmerjahn and Ravetch, 2008). FcγRIII is an activating Fc receptor and found broadly on the surface of myeloid cells and is the only IgG receptor expressed by NK cells (Nimmerjahn and Ravetch, 2008; Ravetch et al., 2001). FcγRIIB, an inhibitory IgG receptor, is the only IgG Fc receptor expressed by B cells. It is also expressed on a variety of myeloid cells, but not expressed by NK cells (Ravetch et al., 2001). On NK cells and myeloid cells, FcγRIII is known to potently mediate antibody-dependent cell-mediated cytotoxicity (ADCC) through binding to IgG1 immune complexes, a process negatively regulated on myeloid cells by concurrent signals through FcγRIIB (Ravetch et al., 2001; Nimmerjahn and Ravetch, 2007; Desai et al., 2007).

Antibodies targeting cell-surface antigens expressed by tumors have shown great promise in eliminating cancer cells (Nimmerjahn and Ravetch, 2007; Boross and Leusen, 2012; Pokrass et al., 2013; Boross et al., 2013; Scott et al., 2012). Part of the efficacy of therapeutic anti-tumor antibodies may be through ADCC (Nimmerjahn and Ravetch, 2007). It has also been suggested that these antibodies may induce CTL responses by targeting tumors to dendritic cells (Signorino et al., 2007; Dhodapkar et al., 2002; Weiner et al., 2009). Indeed, Fc receptor-mediated uptake of antigen-antibody complexes triggers highly efficient presentation of Fc-targeted antigens and induction of T cell responses (Desai et al., 2007; Rafiq et al., 2002; Harbers et al., 2007; Getahun et al., 2004; Regnault et al., 1999).

In all nucleated cells, cytosolic antigens are presented on MHC Class I (MHC-I) molecules. In specialized cells capable of phagocytosis, such as macrophages, endocytosed antigens are presented largely on MHC-II molecules. In contrast, DCs possess the unique ability to cross-present endocytosed antigen to CD8+ T cells via MHC-I. Cross-presentation is critical for the initiation of CD8 T cell responses to intracellular pathogens that do not infect DCs directly (Joffre et al., 2012). Targeting antigens to Fc receptors on DCs also leads to very efficient priming of CD8 T cells (Regnault et al., 1999; Amigorena, 2002; den Haan and Bevan, 2002). The ability to harness this power in cancer therapy would be highly valuable.

### SUMMARY OF THE INVENTION

Thus, in accordance with the present disclosure, there is provided a method of treating cancer in a subject comprising (a) providing a recombinant cancer cell that expresses Ig Fc on its surface; and (b) administering the recombinant cancer cell to the subject. The subject may be a human subject or a non-human mammal. The cancer may be a solid tumor, such as breast cancer, lung cancer, colon cancer, pancreatic cancer, renal cancer, stomach cancer, liver cancer, bone cancer, neural tissue cancer, melanoma, ovarian cancer, testicular cancer, prostate cancer, cervical cancer, vaginal cancer, or bladder cancer. The cancer may be a hematologic cancer, such as a leukemia or lymphoma. The cancer may be recurrent, metastatic and/or multi-drug resistant. The recombinant cancer cell may be autologous to the subject or not autologous to the subject. The method may inhibit metastasis, inhibit primary tumor growth, induce primary tumor regression, reduce tumor burden, or render an unresectable solid tumor resectable.

The recombinant cancer cell may be transformed with an expression construct that expresses the Ig Fc, such as a viral expression construct or a a non-viral expression construct. The expression construct may comprise an inducible, constitutive or tissue selective/specific promoter. The tissue selective/specific promoter may be active in the cancer cell. The Ig Fc molecule may be an IgG Fc molecule, such as an IgG1 Fc molecule. The recombinant cancer cell may be engineered to express one or more heterologous tumor antigens.

The method may further comprise treating the subject with a second cancer therapy, such as surgery, chemotherapy, radiotherapy, gene therapy, toxin therapy, hormone therapy or an immunotherapy. The immunotherapy may comprise treating the subject with a TLR3 ligand or a RIG-I ligand, such as poly I:C. The method may further comprise assessing the genotype and/or phenotype of the cancer prior to treatment. The method may further comprise (i) obtaining, prior to treatment, a cancer cell from the subject; and (ii) engineering the cancer to produce the recombinant cancer cell. The method may further comprise assessing an immune response to the recombinant cancer cell after step (b), such as a CD8+ T cell response.

In another embodiment, there is provided a method of prophylactically treating cancer in a subject comprising (a) providing a recombinant cancer cell that expresses Ig Fc on its surface; and (b) administering the recombinant cancer cell to the subject. The subject may have been determined to be at risk of cancer. The Ig Fc molecule may be an IgG Fc molecule, such as an IgG1 Fc molecule. The method may further comprise administering to the subject a TLR3 ligand or a RIG-I ligand, such as poly I:C.

In still a further embodiment, there is provided a recombinant cancer cell that expresses Ig Fc on its surface. The recombinant cancer cell may comprise a heterologous expression construct encoding the Ig Fc, such as a viral expression construct or a non-viral expression construct. The expression construct may comprise an inducible, constitutive or tissue selective/specific promoter, and the tissue selective/specific promoter may be active in the cancer cell. The Ig Fc molecule may be an IgG Fc molecule, such as an IgG1 Fc molecule. The recombinant cancer cell may be engineered to express one or more heterologous tumor antigens. The recombinant cancer cell may be formulated with a pharmaceutically acceptable buffer or diluent, a preservative, an adjuvant, and/or an immunodulatory compound.

In a further embodiment, there is provided a kit comprising recombinant cancer cell that expresses Ig Fc on its surface, such as an IgG1 Fc molecule. The recombinant cancer cell may be is engineered to express one or more heterologous tumor antigens. The kit may further comprise a pharmaceutically acceptable buffer or diluent, a preservative, an adjuvant, and/or an immunomodulatory compound. The kit may further comprise a device for administering the recombinant cancer cell to a subject.

As used herein the specification, “a” or “an” may mean one or more. As used herein in the claim(s), when used in conjunction with the word “comprising,” the words “a” or “an” may mean one or more than one. As used herein “another” may mean at least a second or more. Other objects, features and advantages of the present disclosure will become apparent from the following detailed description. It should be understood, however, that the detailed description and the specific examples, while indicating preferred embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIGS. 1A-B. Creation of IgG1 Fc expressing tumor cells. (FIG. 1A) A construct expressing IgG1 Fc-transferrin transmembrane region was subcloned into a retroviral vector expressing IRES-GFP. EG7 cells were transduced with retro virus expressing either GFP containing empty vector (EG7-EV) or the IgG1 Fc construct EG7-Fc). (FIG. 1B) Lower panels show staining for GFP (X-axis) and IgG1 (Y axis) in sorted cells that express just the empty vector (left panel) or the mTR-Fc construct (right panel).

FIGS. 2A-D. Dendritic cells exposed to EG7-Fc bearing tumors induce robust activation of antigen specific CD8 T cells. (FIGS. 2A-B) GM-CSF derived BMDCS and C-D, Flt3-ligand derived splenic DCs were co-incubated with either EG7-EV cells or EG7-Fc cells for a period of 12-16 hours. DCs were then sorted on a flow cytometer and were then plated at different concentrations in the presence of either OT-I (FIGS. 2A, 2C) or OT-II (FIGS. 2B, 2D) T cells. After 3 days of culturing, T cell proliferation was measured by ³H thymidine incorporation. Ovalbumin (10 μg/ml) pulsed DCs were used as a control to measure both CD8 (OT-I) and CD4 (OT-II) T cell proliferation. The data are representative of five independent experiments.

FIGS. 3A-B. Dendritic cells exposed to EG7-Fc bearing tumors prime functionally superior CD8 T cells. GM-CSF derived BMDCS co-incubated with either EG7-EV cells or EG7-Fc cells for a period of 12-16 hours. DCs were then sorted on a flow cytometer and were then incubated at 1:10 ratio with OT-I T cells. After 3 days of culturing, CD8 T cells were assessed for intracellular IFN-gamma, TNF-alpha and Granzyme B (FIG. 3A). CD8 T cells were also incubated with target cells (EG7) at the indicated effector:target ratio for a period of 12 hours to measure cytotoxicity (FIG. 3B). The data are representative of two independent experiments.

FIGS. 4A-C. Enhanced CD8 T cell activation can be inhibited by blocking Fc receptors and enhanced by the addition of TLR3 agonist. (FIG. 4A) GM-CSF BMDCs were pre-incubated with a control antibody or a blocking antibody to Fc receptors (clone 2.4G2) before being cultured with either EG7-EV or EG7-Fc cells. After overnight culture, DCs were sorted and plated at different concentrations before addition of OT-I T cells. T cell proliferation was measured after 72 hours as described previously. (FIGS. 4B-C) Experiments performed as previously with the addition of 10 μM poly I:C to tumor-BMDC cultures 8 hours prior to incubation with OT-I cells (FIG. 4B) and OT-II cells (FIG. 4C). The data are representative of three independent experiments.

FIGS. 5A-B. DC-Tumor interaction time is prolonged when tumors express IgG1Fc. (FIG. 5A) A representative bright field and fluorescence image overlay from a 4 hour time-lapsed imaging experiment with DCs co-cultured with GFP expressing cancer cells (green) is given. (FIG. 5B) Interaction times of DCs with tumor cells (±IgG1Fc) were analyzed as described in the materials and methods. The data are representative of two independent experiments and p values were determined by two-tailed unpaired T test.

FIGS. 6A-B. EG7-Fc tumor cells fail to grow in vivo and induce higher CD8 T cell responses. (FIG. 6A) Groups of 15 mice were implanted subcutaneously with 5×10⁵ tumor cells in the flanks. Five mice from each group were sacrificed on days 7, 14 and 21 and tumors were excised and weighed to measure growth. Mice that received empty vector expressing tumor cells grew large tumors by day 30, however mice that received mTR-Fc cells failed to grow detectable tumors at day 30. Both groups had palpable and measurable tumors at days 7 and 14. (FIG. 6B) Draining lymph nodes (inguinal) were harvested and pooled from 5 mice for each group. CD8+ T cells were purified using negative selection and allowed to proliferate on BMDCs that had been cocultured with tumor cells for 12 hours prior and purified by FACS. After 48 hours of culture, CD8 T cell proliferation was measured by ³H thymidine incorporation. The data are representative of three independent experiments and p values were determined by two-tailed unpaired T test.

FIGS. 7A-C. EG7-Fc tumors are functional both as a prophylactic inactivated cell vaccine and as a therapeutic live cell vaccine. (FIG. 7A), Groups of 5 mice were administered either EG7-EV or EG7-Fc cells (5×10⁵, mitomycin C treated) as vaccines in the left flanks. After 14 days, both groups received unmanipulated live EG7 cells subcutaneously in the right flank. Mice were followed longitudinally and tumor volumes were assessed by using Vernier's Calipers. (FIGS. 7B-C) Mice were injected with 5×10⁵ live unmanipulated EG7 tumors into the left flank. Mice (n=15 each group) were then treated with 5×10⁵ of live tumor (EG7-EV or EG7-Fc) or vehicle in the right flank on day 1, 2, 4 and 10. Mice treated with EG7-Fc expressing tumors had significantly smaller primary tumors in the left flank by day 21 than animals treated with vehicle (p<0.05, independent t-test) while animals treated with EG7-EV did not show any diminution in tumor size compared to the vehicle. FIG. 7B shows the raw data, FIG. 7C shows mean tumor volume of each group. The data are representative of two independent experiments.

FIGS. 8A-B. Doubling time is not different between modified tumors. (FIG. 8A) Tumors were plated at 10⁵/ml and counted on day 3. No significant difference was seen in cell turnover rate (replicates of 3 in each group). (FIG. 8B) No significant difference was seen in the incorporation of ³H thymidine into cell cultures over an 18-hour period.

FIGS. 9A-B. Culture of DCs with IgG1 Fc expressing tumor does not result in upregulatio of activation markers on DCs. Tumors were cultured with BMDCs overnight and DCs were stained to measure upregulation of CD86 (FIG. 9A) or CD40 (FIG. 9B). Heat killed (HK) Salmonella typhimurium was used as a positive control for DC maturation.

FIG. 10. DCs cultured with IgG1 Fc expressing tumors do not secrete pro-inflammatory cytokines. Supernatants from BMDCs cultured as described in FIGS. 9A-B were assayed for the presence of IL-6 and IL-12 by quantitative ELISA.

### DETAILED DESCRIPTION

The inventors hypothesized that targeted recognition of tumor cells by dendritic cells/myeloid cells via murine IgG1 Fc, the least inflammatory mouse IgG isotype, would promote efficient CTL responses while minimizing dangerous inflammatory side effects. In this study, the inventors show that tumor cells expressing the Fc portion of murine IgG1 enhance the cross-presentation of a model antigen, and trigger a potent anti-tumor immune response in vitro and in vivo. These and other aspects of the disclosure are discussed below.

### I. IMMUNOGLOBULINS

Antibodies according to the present disclosure may be of any class, but in a particular embodiment, the antibody is an Immunoglobulin G (IgG) antibody isotype. Representing approximately 75% of serum immunoglobulins in humans, IgG is the most abundant antibody isotype found in the circulation. IgG molecules are synthesized and secreted by plasma B cells. There are four IgG subclasses (IgG1, 2, 3, and 4) in humans, named in order of their abundance in serum (IgG1 being the most abundant). The range from having high to no affinity for the Fc receptor.

IgG is the main antibody isotype found in blood and extracellular fluid allowing it to control infection of body tissues. By binding many kinds of pathogens—representing viruses, bacteria, and fungi—IgG protects the body from infection. It does this via several immune mechanisms: IgG-mediated binding of pathogens causes their immobilization and binding together via agglutination; IgG coating of pathogen surfaces (known as opsonization) allows their recognition and ingestion by phagocytic immune cells; IgG activates the classical pathway of the complement system, a cascade of immune protein production that results in pathogen elimination; IgG also binds and neutralizes toxins. IgG also plays an important role in antibody-dependent cell-mediated cytotoxicity (ADCC) and intracellular antibody-mediated proteolysis, in which it binds to TRIM21 (the receptor with greatest affinity to IgG in humans) in order to direct marked virions to the proteasome in the cytosol. IgG is also associated with Type II and Type III Hypersensitivity. IgG antibodies are generated following class switching and maturation of the antibody response and thus participate predominantly in the secondary immune response. IgG is secreted as a monomer that is small in size allowing it to easily perfuse tissues. It is the only isotype that has receptors to facilitate passage through the human placenta. Along with IgA secreted in the breast milk, residual IgG absorbed through the placenta provides the neonate with humoral immunity before its own immune system develops. Colostrum contains a high percentage of IgG, especially bovine colostrum. In individuals with prior immunity to a pathogen, IgG appears about 24-48 hours after antigenic stimulation.

The starting sequences, depending on source, may need to be engineered to effect cell surface expression of an Fc region. The following is a general discussion of relevant techniques for antibody engineering.

Hybridomas may be cultured, then cells lysed, and total RNA extracted. Random hexamers may be used with RT to generate cDNA copies of RNA, and then PCR performed using a multiplex mixture of PCR primers expected to amplify all human variable gene sequences. PCR product can be cloned into pGEM-T Easy vector, then sequenced by automated DNA sequencing using standard vector primers. Recombinant full length IgG antibodies can be generated by subcloning heavy and light chain Fv DNAs from the cloning vector into a Lonza pConIgG1 or pConK2 plasmid vector, transfected into 293 Freestyle cells or Lonza CHO cells, and collected and purified from the CHO cell supernatant.

pCon Vectors' are an easy way to re-express whole or partial antibodies. The constant region vectors are a set of vectors offering a range of immunoglobulin constant region vectors cloned into the pEE vectors. These vectors offer easy construction of full length antibodies with human constant regions and the convenience of the GS System™.

The present disclosure also contemplates isotype modification. By modifying the Fc region to have a different isotype, different functionalities can be achieved. For example, changing to IgG4 can reduce immune effector functions associated with other isotypes.

Modified antibodies may be made by any technique known to those of skill in the art, including expression through standard molecular biological techniques, or the chemical synthesis of polypeptides. Methods for recombinant expression are addressed elsewhere in this document.

### II. CANCER CELL ENGINEERING

Nucleic acids according to the present disclosure will encode antibodies, and in particular Fc regions, optionally linked to other coding or non-coding sequences, and may be used for expression of Fc regions in target cells, i.e., cancer cells. As used in this application, the term “a nucleic acid encoding an antibody or fragment thereof” refers to a nucleic acid molecule that has been isolated free of total cellular nucleic acid. In certain embodiments, the disclosure concerns a nucleic acid encoding an IgG1 Fc region. The cancer cell may be virtually any cancer cell, and may be obtained from a patient for re-introduction (autologous therapy) or may be used in another patient having a similar cancer (heterologous therapy).

Within certain embodiments, expression vectors are employed to express a MUC1-C ligand trap in order to produce and isolate the polypeptide expressed therefrom. In other embodiments, the expression vectors are used in gene therapy. Expression requires that appropriate signals be provided in the vectors, and which include various regulatory elements, such as enhancers/promoters from both viral and mammalian sources that drive expression of the genes of interest in host cells. Elements designed to optimize messenger RNA stability and translatability in host cells also are defined. The conditions for the use of a number of dominant drug selection markers for establishing permanent, stable cell clones expressing the products are also provided, as is an element that links expression of the drug selection markers to expression of the polypeptide.

Throughout this application, the term “expression construct” is meant to include any type of genetic construct containing a nucleic acid coding for a gene product in which part or all of the nucleic acid encoding sequence is capable of being transcribed. The transcript may be translated into a protein, but it need not be. In certain embodiments, expression includes both transcription of a gene and translation of mRNA into a gene product. In other embodiments, expression only includes transcription of the nucleic acid encoding a gene of interest.

The term “vector” is used to refer to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art would be well equipped to construct a vector through standard recombinant techniques, which are described in Sambrook et al. (1989) and Ausubel et al. (1994), both incorporated herein by reference.

The term “expression vector” refers to a vector containing a nucleic acid sequence coding for at least part of a gene product capable of being transcribed. In some cases, RNA molecules are then translated into a protein, polypeptide, or peptide. In other cases, these sequences are not translated, for example, in the production of antisense molecules or ribozymes. Expression vectors can contain a variety of “control sequences,” which refer to nucleic acid sequences necessary for the transcription and possibly translation of an operably linked coding sequence in a particular host organism. In addition to control sequences that govern transcription and translation, vectors and expression vectors may contain nucleic acid sequences that serve other functions as well and are described infra.

A. Regulatory Elements

A “promoter” is a control sequence that is a region of a nucleic acid sequence at which initiation and rate of transcription are controlled. It may contain genetic elements at which regulatory proteins and molecules may bind such as RNA polymerase and other transcription factors. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence. A promoter may or may not be used in conjunction with an “enhancer,” which refers to a cis-acting regulatory sequence involved in the transcriptional activation of a nucleic acid sequence.

A promoter may be one naturally-associated with a gene or sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment.

A recombinant or heterologous enhancer refers also to an enhancer not normally associated with a nucleic acid sequence in its natural environment. Such promoters or enhancers may include promoters or enhancers of other genes, and promoters or enhancers isolated from any other prokaryotic, viral, or eukaryotic cell, and promoters or enhancers not “naturally-occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. No. 4,683,202, U.S. Pat. No. 5,928,906, each incorporated herein by reference). Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the cell type, organelle, and organism chosen for expression. Those of skill in the art of molecular biology generally know the use of promoters, enhancers, and cell type combinations for protein expression, for example, see Sambrook et al. (1989), incorporated herein by reference. The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

Table 1 lists several elements/promoters that may be employed, in the context of the present disclosure, to regulate the expression of a gene. This list is not intended to be exhaustive of all the possible elements involved in the promotion of expression but, merely, to be exemplary thereof. Table 2 provides examples of inducible elements, which are regions of a nucleic acid sequence that can be activated in response to a specific stimulus.

TABLE 1
PROMOTER AND/OR ENHANCER

| Promoter/Enhancer | References |
|---|---|
| Immunoglobulin | Banerji et al., 1983; Gilles et al., 1983; |
| Heavy Chain | Grosschedl et al., 1985; Atchinson et al., 1986, |
| | 1987; Imler et al., 1987; Weinberger et al., 1984; |
| | Kiledjian et al., 1988; Porton et al.; 1990 |
| Immunoglobulin | Queen et al., 1983; Picard et al., 1984 |
| Light Chain | |
| T-Cell Receptor | Luria et al., 1987; Winoto et al., 1989; Redondo |
| | et al.; 1990 |
| HLA DQ a and/or | Sullivan et al., 1987 |
| DQ β | |
| β-Interferon | Goodbourn et al., 1986; Fujita et al., 1987; |
| | Goodbourn et al., 1988 |
| Interleukin-2 | Greene et al., 1989 |
| Interleukin-2 | Greene et al., 1989; Lin et al., 1990 |
| Receptor | |
| MHC Class II 5 | Koch et al., 1989 |
| MHC Class II | Sherman et al., 1989 |
| HLA-DRa | |
| β-Actin | Kawamoto et al., 1988; Ng et al.; 1989 |
| Muscle Creatine | Jaynes et al., 1988; Horlick et al., 1989; Johnson |
| Kinase (MCK) | et al., 1989 |
| Prealbumin | Costa et al., 1988 |
| (Transthyretin) | |
| Elastase I | Ornitz et al., 1987 |
| Metallothionein | Karin et al., 1987; Culotta et al., 1989 |
| (MTII) | |
| Collagenase | Pinkert et al., 1987; Angel et al., 1987 |
| Albumin | Pinkert et al., 1987; Tronche et al., 1989, 1990 |
| α-Fetoprotein | Godbout et al., 1988; Campere et al., 1989 |
| t-Globin | Bodine et al., 1987; Perez-Stable et al., 1990 |
| β-Globin | Trudel et al., 1987 |
| c-fos | Cohen et al., 1987 |
| c-HA-ras | Triesman, 1986; Deschamps et al., 1985 |
| Insulin | Edlund et al., 1985 |
| Neural Cell | Hirsh et al., 1990 |
| Adhesion Molecule | |
| (NCAM) | |
| α₁-Antitrypain | Latimer et al., 1990 |
| H2B (TH2B) Histone | Hwang et al., 1990 |
| Mouse and/or | Ripe et al., 1989 |
| Type I Collagen | |
| Glucose-Regulated | Chang et al., 1989 |
| Proteins (GRP94 | |
| and GRP78) | |
| Rat Growth Hormone | Larsen et al., 1986 |
| Human Serum | Edbrooke et al., 1989 |
| Amyloid A (SAA) | |
| Troponin I (TN I) | Yutzey et al., 1989 |
| Platelet-Derived | Pech et al., 1989 |
| Growth Factor | |
| (PDGF) | |
| Duchenne Muscular | Klamut et al., 1990 |
| Dystrophy | |
| SV40 | Banerji et al., 1981; Moreau et al., 1981; Sleigh et |
| | al., 1985; Firak et al., 1986; Herr et al., 1986; |
| | Imbra et al., 1986; Kadesch et al., 1986; Wang et |
| | al., 1986; Ondek et al., 1987; |
| | Kuhl et al., 1987; Schaffner et al., 1988 |
| Polyoma | Swartzendruber et al., 1975; Vasseur et al., 1980; |
| | Katinka et al., 1980, 1981; Tyndell et al., 1981; |
| | Dandolo et al., 1983; de Villiers et al., 1984; Hen |
| | et al., 1986; Satake et al., 1988; Campbell and |
| | Villarreal, 1988 |
| Retroviruses | Kriegler et al., 1982, 1983; Levinson et al., 1982; |
| | Kriegler et al., 1983, 1984a, b, 1988; Bosze et al., |
| | 1986; Miksicek et al., 1986; Celander et al., 1987; |
| | Thiesen et al., 1988; Celander et al., 1988; Choi |
| | et al., 1988; Reisman et al., 1989 |
| Papilloma Virus | Campo et al., 1983; Lusky et al., 1983; Spandidos |
| | and/or Wilkie, 1983; Spalholz et al., 1985; Lusky |
| | et al., 1986; Cripe et al., 1987; Gloss et al., |
| | 1987; Hirochika et al., 1987; Stephens et al., 1987 |
| | Glue et al., 1988 |
| Hepatitis B Virus | Bulla et al., 1986; Jameel et al., 1986; |
| | Shaul et al., 1987; Spandau et al., 1988; |
| | Vannice et al., 1988 |
| Human | Muesing et al., 1987; Hauber et al., 1988; |
| Immunodeficiency | Jakobovits et al., 1988; Feng et al., 1988; |
| Virus | Takebe et al., 1988; Rosen et al., 1988; Berkhout |
| | et al., 1989; Laspia et al., 1989; Sharp et al., |
| | 1989; Braddock et al., 1989 |
| Cytomegalovirus | Weber et al., 1984; Boshart et al., 1985; Foecking |
| (CMV) | et al., 1986 |
| Gibbon Ape | Holbrook et al., 1987; Quinn et al., 1989 |
| Leukemia Virus | |

TABLE 2
INDUCIBLE ELEMENTS

| Element | Inducer | References |
|---|---|---|
| MT II | Phorbol Ester (TFA) | Palmiter et al., 1982; |
| | Heavy metals | Haslinger et al., 1985; |
| | | Searle et al., 1985; Stuart |
| | | et al., 1985; Imagawa |
| | | et al., 1987, Karin et al., |
| | | 1987; Angel et al., 1987b; |
| | | McNeall et al., 1989 |
| MMTV (mouse | Glucocorticoids | Huang et al., 1981; Lee et |
| mammary | | al., 1981; Majors et al., |
| tumor virus) | | 1983; Chandler et al., 1983; |
| | | Lee et al., 1984; Ponta et |
| | | al., 1985; Sakai et al., 1988 |
| β-Interferon | poly(rI)x | Tavernier et al., 1983 |
| | poly(rc) | |
| Adenovirus 5 E2 | ElA | Imperiale et al., 1984 |
| Collagenase | Phorbol Ester (TPA) | Angel et al., 1987a |
| Stromelysin | Phorbol Ester (TPA) | Angel et al., 1987b |
| SV40 | Phorbol Ester (TPA) | Angel et al., 1987b |
| Murine MX Gene | Interferon, Newcastle | Hug et al., 1988 |
| | Disease Virus | |
| GRP78 Gene | A23187 | Resendez et al., 1988 |
| α-2-Macroglobulin | IL-6 | Kunz et al., 1989 |
| Vimentin | Serum | Rittling et al., 1989 |
| MHC Class I Gene | Interferon | Blanar et al., 1989 |
| H-2κb | | |
| HSP70 | ElA, SV40 Large | Taylor et al., 1989, 1990a, |
| | T Antigen | 1990b |
| Proliferin | Phorbol Ester-TPA | Mordacq et al., 1989 |
| Tumor Necrosis | PMA | Hensel et al., 1989 |
| Factor | | |
| Thyroid Stimulating | Thyroid Hormone | Chatterjee et al., 1989 |
| Hormone α Gene | | |

The identity of tissue-specific promoters or elements, as well as assays to characterize their activity, is well known to those of skill in the art. Examples of such regions include the human LIMK2 gene (Nomoto et al. 1999), the somatostatin receptor 2 gene (Kraus et al., 1998), murine epididymal retinoic acid-binding gene (Lareyre et al., 1999), human CD4 (Zhao-Emonet et al., 1998), mouse alpha2 (XI) collagen (Tsumaki, et al., 1998), DIA dopamine receptor gene (Lee, et al., 1997), insulin-like growth factor II (Wu et al., 1997), human platelet endothelial cell adhesion molecule-1 (Almendro et al., 1996). Tumor specific promoters also will find use in the present disclosure. Some such promoters are set forth in Table 3.

TABLE 3
TISSUE-SPECIFIC PROMOTERS FOR CANCER CELL EXPRESSION

| | Cancers in which | Normal cells in which |
|---|---|---|
| Tissue-specific promoter | promoter is active | promoter is active |
| Carcinoembryonic antigen | Most colorectal carcinomas; | Colonic mucosa; gastric |
| (CEA)* | 50% of lung carcinomas; | mucosa; lung epithelia; |
| | 40-50% of gastric carcinomas; | eccrine sweat glands; |
| | most pancreatic carcinomas; | cells in testes |
| | many breast carcinomas | |
| Prostate-specific antigen | Most prostate carcinomas | Prostate epithelium |
| (PSA) | | |
| Vasoactive intestinal peptide | Majority of non-small cell | Neurons; lymphocytes; mast |
| (VIP) | lung cancers | cells; eosinophils |
| Surfactant protein A (SP-A) | Many lung adenocarcinomas | Type II pneumocytes; Clara |
| | cells | |
| Human achaete-scute | Most small cell lung cancers | Neuroendocrine cells in lung |
| homolog (hASH) | | |
| Mucin-1 (MUC1)** | Most adenocarcinomas | Glandular epithelial cells in |
| | (originating from any tissue) | breast and in respiratory, |
| | | gastrointestinal, and |
| | | genitourinary tracts |
| Alpha-fetoprotein | Most hepatocellular | Hepatocytes (under certain |
| | carcinomas; possibly many | conditions); testis |
| | testicular cancers | |
| Albumin | Most hepatocellular | Hepatocytes |
| | carcinomas | |
| Tyrosinase | Most melanomas | Melanocytes; astrocytes; |
| | | Schwann cells; some neurons |
| Tyrosine-binding protein | Most melanomas | Melanocytes; astrocytes, |
| (TRP) | | Schwann cells; some neurons |
| Keratin 14 | Presumably many squamous | Keratinocytes |
| | cell carcinomas (e.g., Head | |
| | and neck cancers) | |
| EBV LD-2 | Many squamous cell | Keratinocytes of upper |
| | carcinomas of head and neck | digestive |
| | | Keratinocytes of |
| | | upper digestive tract |
| Glial fibrillary acidic protein | Many astrocytomas | Astrocytes |
| (GFAP) | | |
| Myelin basic protein (MBP) | Many gliomas | Oligodendrocytes |
| Testis-specific angiotensin- | Possibly many testicular | Spermatazoa |
| converting enzyme (Testis- | cancers | |
| specific ACE) | | |
| Osteocalcin | Possibly many osteosarcomas | Osteoblasts |
| E2F-regulated promoter | Almost all cancers | Proliferating cells |
| HLA-G | Many colorectal carcinomas; | Lymphocytes; |
| | many melanomas; possibly | monocytes; |
| | many other cancers | spermatocytes; |
| | | trophoblast |
| FasL | Most melanomas; many | Activated leukocytes: |
| | pancreatic carcinomas; most | neurons; endothelial |
| | astrocytomas possibly many | cells; keratinocytes; |
| | other cancers | cells in |
| | | immunoprivileged |
| | | tissues; some cells in |
| | | lungs, ovaries, liver, |
| | | and prostate |
| Myc-regulated promoter | Most lung carcinomas (both | Proliferating cells |
| | small cell and non-small cell); | (only some cell-types): |
| | most colorectal carcinomas | mammary epithelial |
| | | cells (including non- |
| | | proliferating) |
| MAGE-1 | Many melanomas; some non- | Testis |
| | small cell lung carcinomas; | |
| | some breast carcinomas | |
| VEGF | 70% of all cancers | Cells at sites of |
| | (constitutive overexpression in | neovascularization |
| | many cancers) | (but unlike in tumors, |
| | | expression is transient, |
| | | less strong, and never |
| | | constitutive) |
| bFGF | Presumably many different | Cells at sites of |
| | cancers, since bFGF | ischemia (but unlike |
| | expression is induced by | tumors, expression is |
| | ischemic conditions | transient, less strong, |
| | | and never constitutive) |
| COX-2 | Most colorectal carcinomas; | Cells at sites of |
| | many lung carcinomas; | inflammation |
| | possibly many other cancers | |
| IL-10 | Most colorectal carcinomas; | Leukocytes |
| | many lung carcinomas; many | |
| | squamous cell carcinomas of | |
| | head and neck; possibly many | |
| | other cancers | |
| GRP78/BiP | Presumably many different | Cells at sites of |
| | cancers, since GRP7S | ishemia |
| | expression is induced by | |
| | tumor-specific conditions | |
| CarG elements from Egr-1 | Induced by ionization | Cells exposed to |
| | radiation, so conceivably most | ionizing radiation; |
| | tumors upon irradiation | leukocytes |

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

B. Multi-Purpose Cloning Sites

Vectors can include a multiple cloning site (MCS), which is a nucleic acid region that contains multiple restriction enzyme sites, any of which can be used in conjunction with standard recombinant technology to digest the vector. See Carbonelli et al., 1999, Levenson et al., 1998, and Cocea, 1997, incorporated herein by reference. “Restriction enzyme digestion” refers to catalytic cleavage of a nucleic acid molecule with an enzyme that functions only at specific locations in a nucleic acid molecule. Many of these restriction enzymes are commercially available. Use of such enzymes is widely understood by those of skill in the art. Frequently, a vector is linearized or fragmented using a restriction enzyme that cuts within the MCS to enable exogenous sequences to be ligated to the vector. “Ligation” refers to the process of forming phosphodiester bonds between two nucleic acid fragments, which may or may not be contiguous with each other. Techniques involving restriction enzymes and ligation reactions are well known to those of skill in the art of recombinant technology.

C. Splicing Sites

Most transcribed eukaryotic RNA molecules will undergo RNA splicing to remove introns from the primary transcripts. Vectors containing genomic eukaryotic sequences may require donor and/or acceptor splicing sites to ensure proper processing of the transcript for protein expression (see Chandler et al., 1997, herein incorporated by reference).

D. Termination Signals

The vectors or constructs of the present disclosure will generally comprise at least one termination signal. A “termination signal” or “terminator” is comprised of the DNA sequences involved in specific termination of an RNA transcript by an RNA polymerase. Thus, in certain embodiments a termination signal that ends the production of an RNA transcript is contemplated. A terminator may be necessary in vivo to achieve desirable message levels.

In eukaryotic systems, the terminator region may also comprise specific DNA sequences that permit site-specific cleavage of the new transcript so as to expose a polyadenylation site. This signals a specialized endogenous polymerase to add a stretch of about 200 A residues (polyA) to the 3′ end of the transcript. RNA molecules modified with this polyA tail appear to more stable and are translated more efficiently. Thus, in other embodiments involving eukaryotes, it is preferred that that terminator comprises a signal for the cleavage of the RNA, and it is more preferred that the terminator signal promotes polyadenylation of the message. The terminator and/or polyadenylation site elements can serve to enhance message levels and/or to minimize read through from the cassette into other sequences.

Terminators contemplated for use in the disclosure include any known terminator of transcription described herein or known to one of ordinary skill in the art, including but not limited to, for example, the termination sequences of genes, such as for example the bovine growth hormone terminator or viral termination sequences, such as for example the SV40 terminator. In certain embodiments, the termination signal may be a lack of transcribable or translatable sequence, such as due to a sequence truncation.

E. Polyadenylation Signals

In expression, particularly eukaryotic expression, one will typically include a polyadenylation signal to effect proper polyadenylation of the transcript. The nature of the polyadenylation signal is not believed to be crucial to the successful practice of the disclosure, and/or any such sequence may be employed. Preferred embodiments include the SV40 polyadenylation signal and/or the bovine growth hormone polyadenylation signal, convenient and/or known to function well in various target cells. Polyadenylation may increase the stability of the transcript or may facilitate cytoplasmic transport.

F. Origins of Replication

In order to propagate a vector in a host cell, it may contain one or more origins of replication sites (often termed “ori”), which is a specific nucleic acid sequence at which replication is initiated. Alternatively an autonomously replicating sequence (ARS) can be employed if the host cell is yeast.

G. Selectable and Screenable Markers

In certain embodiments of the disclosure, cells containing a nucleic acid construct of the present disclosure may be identified in vitro or in vivo by including a marker in the expression vector. Such markers would confer an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selectable markers. In addition to markers conferring a phenotype that allows for the discrimination of transformants based on the implementation of conditions, other types of markers including screenable markers such as GFP, whose basis is colorimetric analysis, are also contemplated. Alternatively, screenable enzymes such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selectable and screenable markers are well known to one of skill in the art.

H. Viral Vectors

The capacity of certain viral vectors to efficiently infect or enter cells, to integrate into a host cell genome and stably express viral genes, have led to the development and application of a number of different viral vector systems (Robbins et al., 1998). Viral systems are currently being developed for use as vectors for ex vivo and in vivo gene transfer. For example, adenovirus, herpes-simplex virus, retrovirus and adeno-associated virus vectors are being evaluated currently for treatment of diseases such as cancer, cystic fibrosis, Gaucher disease, renal disease and arthritis (Robbins and Ghivizzani, 1998; Imai et al., 1998; U.S. Pat. No. 5,670,488). The various viral vectors described below, present specific advantages and disadvantages, depending on the particular gene-therapeutic application.

Adenoviral Vectors.

In particular embodiments, an adenoviral expression vector is contemplated for the delivery of expression constructs. “Adenovirus expression vector” is meant to include those constructs containing adenovirus sequences sufficient to (a) support packaging of the construct and (b) to ultimately express a tissue or cell-specific construct that has been cloned therein.

Adenoviruses comprise linear, double-stranded DNA, with a genome ranging from 30 to 35 kb in size (Reddy et al., 1998; Morrison et al., 1997; Chillon et al., 1999). An adenovirus expression vector according to the present disclosure comprises a genetically engineered form of the adenovirus. Advantages of adenoviral gene transfer include the ability to infect a wide variety of cell types, including non-dividing cells, a mid-sized genome, ease of manipulation, high infectivity and the ability to be grown to high titers (Wilson, 1996). Further, adenoviral infection of host cells does not result in chromosomal integration because adenoviral DNA can replicate in an episomal manner, without potential genotoxicity associated with other viral vectors. Adenoviruses also are structurally stable (Marienfeld et al., 1999) and no genome rearrangement has been detected after extensive amplification (Parks et al., 1997; Bett et al., 1993).

Salient features of the adenovirus genome are an early region (E1, E2, E3 and E4 genes), an intermediate region (pIX gene, Iva2 gene), a late region (L1, L2, L3, L4 and L5 genes), a major late promoter (MLP), inverted-terminal-repeats (ITRs) and a iv sequence (Zheng, et al., 1999; Robbins et al., 1998; Graham and Prevec, 1995). The early genes E1, E2, E3 and E4 are expressed from the virus after infection and encode polypeptides that regulate viral gene expression, cellular gene expression, viral replication, and inhibition of cellular apoptosis. Further on during viral infection, the MLP is activated, resulting in the expression of the late (L) genes, encoding polypeptides required for adenovirus encapsidation. The intermediate region encodes components of the adenoviral capsid. Adenoviral inverted terminal repeats (ITRs; 100-200 bp in length), are cis elements, and function as origins of replication and are necessary for viral DNA replication. The iv sequence is required for the packaging of the adenoviral genome.

A common approach for generating adenoviruses for use as a gene transfer vectors is the deletion of the E1 gene (E1⁻), which is involved in the induction of the E2, E3 and E4 promoters (Graham and Prevec, 1995). Subsequently, a therapeutic gene or genes can be inserted recombinantly in place of the E1 gene, wherein expression of the therapeutic gene(s) is driven by the E1 promoter or a heterologous promoter. The E1⁻, replication-deficient virus is then proliferated in a “helper” cell line that provides the E1 polypeptides in trans (e.g., the human embryonic kidney cell line 293). Thus, in the present disclosure it may be convenient to introduce the transforming construct at the position from which the E1-coding sequences have been removed. However, the position of insertion of the construct within the adenovirus sequences is not critical to the disclosure. Alternatively, the E3 region, portions of the E4 region or both may be deleted, wherein a heterologous nucleic acid sequence under the control of a promoter operable in eukaryotic cells is inserted into the adenovirus genome for use in gene transfer (U.S. Pat. No. 5,670,488; U.S. Pat. No. 5,932,210, each specifically incorporated herein by reference).

Although adenovirus based vectors offer several unique advantages over other vector systems, they often are limited by vector immunogenicity, size constraints for insertion of recombinant genes and low levels of replication. The preparation of a recombinant adenovirus vector deleted of all open reading frames, comprising a full length dystrophin gene and the terminal repeats required for replication (Haecker et al., 1996) offers some potentially promising advantages to the above mentioned adenoviral shortcomings. The vector was grown to high titer with a helper virus in 293 cells and was capable of efficiently transducing dystrophin in mdx mice, in myotubes in vitro and muscle fibers in vivo. Helper-dependent viral vectors are discussed below.

A major concern in using adenoviral vectors is the generation of a replication-competent virus during vector production in a packaging cell line or during gene therapy treatment of an individual. The generation of a replication-competent virus could pose serious threat of an unintended viral infection and pathological consequences for the patient. Armentano et al. (1990), describe the preparation of a replication-defective adenovirus vector, claimed to eliminate the potential for the inadvertent generation of a replication-competent adenovirus (U.S. Pat. No. 5,824,544, specifically incorporated herein by reference). The replication-defective adenovirus method comprises a deleted E1 region and a relocated protein IX gene, wherein the vector expresses a heterologous, mammalian gene.

Other than the requirement that the adenovirus vector be replication defective, or at least conditionally defective, the nature of the adenovirus vector is not believed to be crucial to the successful practice of the disclosure. The adenovirus may be of any of the 42 different known serotypes and/or subgroups A-F. Adenovirus type 5 of subgroup C is the preferred starting material in order to obtain the conditional replication-defective adenovirus vector for use in the present disclosure. This is because adenovirus type 5 is a human adenovirus about which a great deal of biochemical and genetic information is known, and it has historically been used for most constructions employing adenovirus as a vector.

As stated above, the typical vector according to the present disclosure is replication defective and will not have an adenovirus E1 region. Adenovirus growth and manipulation is known to those of skill in the art, and exhibits broad host range in vitro and in vivo (U.S. Pat. No. 5,670,488; U.S. Pat. No. 5,932,210; U.S. Pat. No. 5,824,544). This group of viruses can be obtained in high titers, e.g., 10⁹ to 10¹¹ plaque-forming units per ml, and they are highly infective. The life cycle of adenovirus does not require integration into the host cell genome. The foreign genes delivered by adenovirus vectors are episomal and, therefore, have low genotoxicity to host cells. Many experiments, innovations, preclinical studies and clinical trials are currently under investigation for the use of adenoviruses as gene delivery vectors. For example, adenoviral gene delivery-based gene therapies are being developed for liver diseases (Han et al., 1999), psychiatric diseases (Lesch, 1999), neurological diseases (Smith, 1998; Hermens and Verhaagen, 1998), coronary diseases (Feldman et al., 1996), muscular diseases (Petrof, 1998), gastrointestinal diseases (Wu, 1998) and various cancers such as colorectal (Fujiwara and Tanaka, 1998; Dorai et al., 1999), pancreatic, bladder (Irie et al., 1999), head and neck (Blackwell et al., 1999), breast (Stewart et al., 1999), lung (Batra et al., 1999) and ovarian (Vanderkwaak et al., 1999).

Retroviral Vectors.

In certain embodiments of the disclosure, the uses of retroviruses for gene delivery are contemplated. Retroviruses are RNA viruses comprising an RNA genome. When a host cell is infected by a retrovirus, the genomic RNA is reverse transcribed into a DNA intermediate which is integrated into the chromosomal DNA of infected cells. This integrated DNA intermediate is referred to as a provirus. A particular advantage of retroviruses is that they can stably infect dividing cells with a gene of interest (e.g., a therapeutic gene) by integrating into the host DNA, without expressing immunogenic viral proteins. Theoretically, the integrated retroviral vector will be maintained for the life of the infected host cell, expressing the gene of interest.

The retroviral genome and the proviral DNA have three genes: gag, pol, and env, which are flanked by two long terminal repeat (LTR) sequences. The gag gene encodes the internal structural (matrix, capsid, and nucleocapsid) proteins; the pol gene encodes the RNA-directed DNA polymerase (reverse transcriptase) and the env gene encodes viral envelope glycoproteins. The 5′ and 3′ LTRs serve to promote transcription and polyadenylation of the virion RNAs. The LTR contains all other cis-acting sequences necessary for viral replication.

A recombinant retrovirus of the present disclosure may be genetically modified in such a way that some of the structural, infectious genes of the native virus have been removed and replaced instead with a nucleic acid sequence to be delivered to a target cell (U.S. Pat. No. 5,858,744; U.S. Pat. No. 5,739,018, each incorporated herein by reference). After infection of a cell by the virus, the virus injects its nucleic acid into the cell and the retrovirus genetic material can integrate into the host cell genome. The transferred retrovirus genetic material is then transcribed and translated into proteins within the host cell. As with other viral vector systems, the generation of a replication-competent retrovirus during vector production or during therapy is a major concern. Retroviral vectors suitable for use in the present disclosure are generally defective retroviral vectors that are capable of infecting the target cell, reverse transcribing their RNA genomes, and integrating the reverse transcribed DNA into the target cell genome, but are incapable of replicating within the target cell to produce infectious retroviral particles (e.g., the retroviral genome transferred into the target cell is defective in gag, the gene encoding virion structural proteins, and/or in pol, the gene encoding reverse transcriptase). Thus, transcription of the provirus and assembly into infectious virus occurs in the presence of an appropriate helper virus or in a cell line containing appropriate sequences enabling encapsidation without coincident production of a contaminating helper virus.

The growth and maintenance of retroviruses is known in the art (U.S. Pat. No. 5,955,331; U.S. Pat. No. 5,888,502, each specifically incorporated herein by reference). Nolan et al. describe the production of stable high titre, helper-free retrovirus comprising a heterologous gene (U.S. Pat. No. 5,830,725, specifically incorporated herein by reference). Methods for constructing packaging cell lines useful for the generation of helper-free recombinant retroviruses with amphoteric or ecotrophic host ranges, as well as methods of using the recombinant retroviruses to introduce a gene of interest into eukaryotic cells in vivo and in vitro are contemplated in the present disclosure (U.S. Pat. No. 5,955,331).

Currently, the majority of all clinical trials for vector-mediated gene delivery use murine leukemia virus (MLV)-based retroviral vector gene delivery (Robbins et al., 1998; Miller et al., 1993). Disadvantages of retroviral gene delivery include a requirement for ongoing cell division for stable infection and a coding capacity that prevents the delivery of large genes. However, recent development of vectors such as lentivirus (e.g., HIV), simian immunodeficiency virus (SIV) and equine infectious-anemia virus (EIAV), which can infect certain non-dividing cells, potentially allow the in vivo use of retroviral vectors for gene therapy applications (Amado and Chen, 1999; Klimatcheva et al., 1999; White et al., 1999; Case et al., 1999). For example, HIV-based vectors have been used to infect non-dividing cells such as neurons (Miyatake et al., 1999), islets (Leibowitz et al., 1999) and muscle cells (Johnston et al., 1999). The therapeutic delivery of genes via retroviruses are currently being assessed for the treatment of various disorders such as inflammatory disease (Moldawer et al., 1999), AIDS (Amado and Chen, 1999; Engel and Kohn, 1999), cancer (Clay et al., 1999), cerebrovascular disease (Weihl et al., 1999) and hemophilia (Kay, 1998).

Herpesviral Vectors.

Herpes simplex virus (HSV) type I and type II contain a double-stranded, linear DNA genome of approximately 150 kb, encoding 70-80 genes. Wild type HSV are able to infect cells lytically and to establish latency in certain cell types (e.g., neurons). Similar to adenovirus, HSV also can infect a variety of cell types including muscle (Yeung et al., 1999), ear (Derby et al., 1999), eye (Kaufman et al., 1999), tumors (Yoon et al., 1999; Howard et al., 1999), lung (Kohut et al., 1998), neuronal (Garrido et al., 1999; Lachmann and Efstathiou, 1999), liver (Miytake et al., 1999; Kooby et al., 1999) and pancreatic islets (Rabinovitch et al., 1999).

HSV viral genes are transcribed by cellular RNA polymerase II and are temporally regulated, resulting in the transcription and subsequent synthesis of gene products in roughly three discernable phases or kinetic classes. These phases of genes are referred to as the Immediate Early (IE) or α genes, Early (E) or β genes and Late (L) or γ genes. Immediately following the arrival of the genome of a virus in the nucleus of a newly infected cell, the IE genes are transcribed. The efficient expression of these genes does not require prior viral protein synthesis. The products of IE genes are required to activate transcription and regulate the remainder of the viral genome.

For use in therapeutic gene delivery, HSV must be rendered replication-defective. Protocols for generating replication-defective HSV helper virus-free cell lines have been described (U.S. Pat. No. 5,879,934; U.S. Pat. No. 5,851,826, each specifically incorporated herein by reference in its entirety). One IE protein, ICP4, also known as α4 or Vmw175, is absolutely required for both virus infectivity and the transition from IE to later transcription. Thus, due to its complex, multifunctional nature and central role in the regulation of HSV gene expression, ICP4 has typically been the target of HSV genetic studies.

Phenotypic studies of HSV viruses deleted of ICP4 indicate that such viruses will be potentially useful for gene transfer purposes (Krisky et al., 1998a). One property of viruses deleted for ICP4 that makes them desirable for gene transfer is that they only express the five other IE genes: ICP0, ICP6, ICP27, ICP22 and ICP47 (DeLuca et al., 1985), without the expression of viral genes encoding proteins that direct viral DNA synthesis, as well as the structural proteins of the virus. This property is desirable for minimizing possible deleterious effects on host cell metabolism or an immune response following gene transfer. Further deletion of IE genes ICP22 and ICP27, in addition to ICP4, substantially improve reduction of HSV cytotoxicity and prevented early and late viral gene expression (Krisky et al., 1998b).

The therapeutic potential of HSV in gene transfer has been demonstrated in various in vitro model systems and in vivo for diseases such as Parkinson's (Yamada et al., 1999), retinoblastoma (Hayashi et al., 1999), intracerebral and intradermal tumors (Moriuchi et al., 1998), B-cell malignancies (Suzuki et al., 1998), ovarian cancer (Wang et al., 1998) and Duchenne muscular dystrophy (Huard et al., 1997).

Adeno-Associated Viral Vectors.

Adeno-associated virus (AAV), a member of the parvovirus family, is a human virus that is increasingly being used for gene delivery therapeutics. AAV has several advantageous features not found in other viral systems. First, AAV can infect a wide range of host cells, including non-dividing cells. Second, AAV can infect cells from different species. Third, AAV has not been associated with any human or animal disease and does not appear to alter the biological properties of the host cell upon integration. For example, it is estimated that 80-85% of the human population has been exposed to AAV. Finally, AAV is stable at a wide range of physical and chemical conditions which lends itself to production, storage and transportation requirements.

The AAV genome is a linear, single-stranded DNA molecule containing 4681 nucleotides. The AAV genome generally comprises an internal non-repeating genome flanked on each end by inverted terminal repeats (ITRs) of approximately 145 bp in length. The ITRs have multiple functions, including origins of DNA replication, and as packaging signals for the viral genome. The internal non-repeated portion of the genome includes two large open reading frames, known as the AAV replication (rep) and capsid (cap) genes. The rep and cap genes code for viral proteins that allow the virus to replicate and package the viral genome into a virion. A family of at least four viral proteins is expressed from the AAV rep region, Rep 78, Rep 68, Rep 52, and Rep 40, named according to their apparent molecular weight. The AAV cap region encodes at least three proteins, VP1, VP2, and VP3.

AAV is a helper-dependent virus requiring co-infection with a helper virus (e.g., adenovirus, herpesvirus or vaccinia) in order to form AAV virions. In the absence of co-infection with a helper virus, AAV establishes a latent state in which the viral genome inserts into a host cell chromosome, but infectious virions are not produced. Subsequent infection by a helper virus “rescues” the integrated genome, allowing it to replicate and package its genome into infectious AAV virions. Although AAV can infect cells from different species, the helper virus must be of the same species as the host cell (e.g., human AAV will replicate in canine cells co-infected with a canine adenovirus).

AAV has been engineered to deliver genes of interest by deleting the internal non-repeating portion of the AAV genome and inserting a heterologous gene between the ITRs. The heterologous gene may be functionally linked to a heterologous promoter (constitutive, cell-specific, or inducible) capable of driving gene expression in target cells. To produce infectious recombinant AAV (rAAV) containing a heterologous gene, a suitable producer cell line is transfected with a rAAV vector containing a heterologous gene. The producer cell is concurrently transfected with a second plasmid harboring the AAV rep and cap genes under the control of their respective endogenous promoters or heterologous promoters. Finally, the producer cell is infected with a helper virus.

Once these factors come together, the heterologous gene is replicated and packaged as though it were a wild-type AAV genome. When target cells are infected with the resulting rAAV virions, the heterologous gene enters and is expressed in the target cells. Because the target cells lack the rep and cap genes and the adenovirus helper genes, the rAAV cannot further replicate, package or form wild-type AAV.

The use of helper virus, however, presents a number of problems. First, the use of adenovirus in a rAAV production system causes the host cells to produce both rAAV and infectious adenovirus. The contaminating infectious adenovirus can be inactivated by heat treatment (56° C. for 1 hour). Heat treatment, however, results in approximately a 50% drop in the titer of functional rAAV virions. Second, varying amounts of adenovirus proteins are present in these preparations. For example, approximately 50% or greater of the total protein obtained in such rAAV virion preparations is free adenovirus fiber protein. If not completely removed, these adenovirus proteins have the potential of eliciting an immune response from the patient. Third, AAV vector production methods which employ a helper virus require the use and manipulation of large amounts of high titer infectious helper virus, which presents a number of health and safety concerns, particularly in regard to the use of a herpesvirus. Fourth, concomitant production of helper virus particles in rAAV virion producing cells diverts large amounts of host cellular resources away from rAAV virion production, potentially resulting in lower rAAV virion yields.

Lentiviral Vectors.

Lentiviruses are complex retroviruses, which, in addition to the common retroviral genes gag, pol, and env, contain other genes with regulatory or structural function. The higher complexity enables the virus to modulate its life cycle, as in the course of latent infection. Some examples of lentivirus include the Human Immunodeficiency Viruses: HIV-1, HIV-2 and the Simian Immunodeficiency Virus: SIV. Lentiviral vectors have been generated by multiply attenuating the HIV virulence genes, for example, the genes env, vif, vpr, vpu and nef are deleted making the vector biologically safe.

Recombinant lentiviral vectors are capable of infecting non-dividing cells and can be used for both in vivo and ex vivo gene transfer and expression of nucleic acid sequences. The lentiviral genome and the proviral DNA have the three genes found in retroviruses: gag, pol and env, which are flanked by two long terminal repeat (LTR) sequences. The gag gene encodes the internal structural (matrix, capsid and nucleocapsid) proteins; the pol gene encodes the RNA-directed DNA polymerase (reverse transcriptase), a protease and an integrase; and the env gene encodes viral envelope glycoproteins. The 5′ and 3′ LTR's serve to promote transcription and polyadenylation of the virion RNA's. The LTR contains all other cis-acting sequences necessary for viral replication. Lentiviruses have additional genes including vif, vpr, tat, rev, vpu, nef and vpx.

Adjacent to the 5′ LTR are sequences necessary for reverse transcription of the genome (the tRNA primer binding site) and for efficient encapsidation of viral RNA into particles (the Psi site). If the sequences necessary for encapsidation (or packaging of retroviral RNA into infectious virions) are missing from the viral genome, the cis defect prevents encapsidation of genomic RNA. However, the resulting mutant remains capable of directing the synthesis of all virion proteins.

Lentiviral vectors are known in the art, see Naldini et al., (1996); Zufferey et al., (1997); U.S. Pat. Nos. 6,013,516; and 5,994,136. In general, the vectors are plasmid-based or virus-based, and are configured to carry the essential sequences for incorporating foreign nucleic acid, for selection and for transfer of the nucleic acid into a host cell. The gag, pol and env genes of the vectors of interest also are known in the art. Thus, the relevant genes are cloned into the selected vector and then used to transform the target cell of interest.

Recombinant lentivirus capable of infecting a non-dividing cell wherein a suitable host cell is transfected with two or more vectors carrying the packaging functions, namely gag, pol and env, as well as rev and tat is described in U.S. Pat. No. 5,994,136, incorporated herein by reference. This describes a first vector that can provide a nucleic acid encoding a viral gag and a pol gene and another vector that can provide a nucleic acid encoding a viral env to produce a packaging cell. Introducing a vector providing a heterologous gene, such as the STAT-1α gene in this disclosure, into that packaging cell yields a producer cell which releases infectious viral particles carrying the foreign gene of interest. The env preferably is an amphotropic envelope protein which allows transduction of cells of human and other species.

One may target the recombinant virus by linkage of the envelope protein with an antibody or a particular ligand for targeting to a receptor of a particular cell-type. By inserting a sequence (including a regulatory region) of interest into the viral vector, along with another gene which encodes the ligand for a receptor on a specific target cell, for example, the vector is now target-specific.

The vector providing the viral env nucleic acid sequence is associated operably with regulatory sequences, e.g., a promoter or enhancer. The regulatory sequence can be any eukaryotic promoter or enhancer, including for example, the Moloney murine leukemia virus promoter-enhancer element, the human cytomegalovirus enhancer or the vaccinia P7.5 promoter. In some cases, such as the Moloney murine leukemia virus promoter-enhancer element, the promoter-enhancer elements are located within or adjacent to the LTR sequences.

The heterologous or foreign nucleic acid sequence, such as the STAT-1α encoding polynucleotide sequence herein, is linked operably to a regulatory nucleic acid sequence. Preferably, the heterologous sequence is linked to a promoter, resulting in a chimeric gene. The heterologous nucleic acid sequence may also be under control of either the viral LTR promoter-enhancer signals or of an internal promoter, and retained signals within the retroviral LTR can still bring about efficient expression of the transgene. Marker genes may be utilized to assay for the presence of the vector, and thus, to confirm infection and integration. The presence of a marker gene ensures the selection and growth of only those host cells which express the inserts. Typical selection genes encode proteins that confer resistance to antibiotics and other toxic substances, e.g., histidinol, puromycin, hygromycin, neomycin, methotrexate, etc., and cell surface markers.

The vectors are introduced via transfection or infection into the packaging cell line. The packaging cell line produces viral particles that contain the vector genome. Methods for transfection or infection are well known by those of skill in the art. After cotransfection of the packaging vectors and the transfer vector to the packaging cell line, the recombinant virus is recovered from the culture media and titered by standard methods used by those of skill in the art. Thus, the packaging constructs can be introduced into human cell lines by calcium phosphate transfection, lipofection or electroporation, generally together with a dominant selectable marker, such as neo, DHFR, Gln synthetase or ADA, followed by selection in the presence of the appropriate drug and isolation of clones. The selectable marker gene can be linked physically to the packaging genes in the construct.

Lentiviral transfer vectors Naldini et al. (1996), have been used to infect human cells growth-arrested in vitro and to transduce neurons after direct injection into the brain of adult rats. The vector was efficient at transferring marker genes in vivo into the neurons and long term expression in the absence of detectable pathology was achieved. Animals analyzed ten months after a single injection of the vector showed no decrease in the average level of transgene expression and no sign of tissue pathology or immune reaction (Blomer et al., 1997). Thus, in the present disclosure, one may graft or transplant cells infected with the recombinant lentivirus ex vivo, or infect cells in vivo.

Other Viral Vectors.

The development and utility of viral vectors for gene delivery is constantly improving and evolving. Other viral vectors such as poxvirus; e.g., vaccinia virus (Gnant et al., 1999; Gnant et al., 1999), alpha virus; e.g., sindbis virus, Semliki forest virus (Lundstrom, 1999), reovirus (Coffey et al., 1998) and influenza A virus (Neumann et al., 1999) are contemplated for use in the present disclosure and may be selected according to the requisite properties of the target system.

In certain embodiments, vaccinia viral vectors are contemplated for use in the present disclosure. Vaccinia virus is a particularly useful eukaryotic viral vector system for expressing heterologous genes. For example, when recombinant vaccinia virus is properly engineered, the proteins are synthesized, processed and transported to the plasma membrane. Vaccinia viruses as gene delivery vectors have recently been demonstrated to transfer genes to human tumor cells, e.g., EMAP-II (Gnant et al., 1999), inner ear (Derby et al., 1999), glioma cells, e.g., p53 (Timiryasova et al., 1999) and various mammalian cells, e.g., P450 (U.S. Pat. No. 5,506,138). The preparation, growth and manipulation of vaccinia viruses are described in U.S. Pat. No. 5,849,304 and U.S. Pat. No. 5,506,138 (each specifically incorporated herein by reference).

In other embodiments, sindbis viral vectors are contemplated for use in gene delivery. Sindbis virus is a species of the alphavirus genus (Garoff and Li, 1998) which includes such important pathogens as Venezuelan, Western and Eastern equine encephalitis viruses (Sawai et al., 1999; Mastrangelo et al., 1999). In vitro, sindbis virus infects a variety of avian, mammalian, reptilian, and amphibian cells. The genome of sindbis virus consists of a single molecule of single-stranded RNA, 11,703 nucleotides in length. The genomic RNA is infectious, is capped at the 5′ terminus and polyadenylated at the 3′ terminus, and serves as mRNA. Translation of a vaccinia virus 26S mRNA produces a polyprotein that is cleaved co- and post-translationally by a combination of viral and presumably host-encoded proteases to give the three virus structural proteins, a capsid protein (C) and the two envelope glycoproteins (E1 and PE2, precursors of the virion E2).

Three features of sindbis virus suggest that it would be a useful vector for the expression of heterologous genes. First, its wide host range, both in nature and in the laboratory. Second, gene expression occurs in the cytoplasm of the host cell and is rapid and efficient. Third, temperature-sensitive mutations in RNA synthesis are available that may be used to modulate the expression of heterologous coding sequences by simply shifting cultures to the non-permissive temperature at various time after infection. The growth and maintenance of sindbis virus is known in the art (U.S. Pat. No. 5,217,879, specifically incorporated herein by reference).

Chimeric Viral Vectors.

Chimeric or hybrid viral vectors are being developed for use in therapeutic gene delivery and are contemplated for use in the present disclosure. Chimeric poxviral/retroviral vectors (Holzer et al., 1999), adenoviral/retroviral vectors (Feng et al., 1997; Bilbao et al., 1997; Caplen et al., 1999) and adenoviral/adeno-associated viral vectors (Fisher et al., 1996; U.S. Pat. No. 5,871,982) have been described.

These “chimeric” viral gene transfer systems can exploit the favorable features of two or more parent viral species. For example, Wilson et al., provide a chimeric vector construct which comprises a portion of an adenovirus, AAV 5′ and 3′ ITR sequences and a selected transgene, described below (U.S. Pat. No. 5,871,983, specifically incorporate herein by reference).

The adenovirus/AAV chimeric virus uses adenovirus nucleic acid sequences as a shuttle to deliver a recombinant AAV/transgene genome to a target cell. The adenovirus nucleic acid sequences employed in the hybrid vector can range from a minimum sequence amount, which requires the use of a helper virus to produce the hybrid virus particle, to only selected deletions of adenovirus genes, which deleted gene products can be supplied in the hybrid viral production process by a selected packaging cell. At a minimum, the adenovirus nucleic acid sequences employed in the pAdA shuttle vector are adenovirus genomic sequences from which all viral genes are deleted and which contain only those adenovirus sequences required for packaging adenoviral genomic DNA into a preformed capsid head. More specifically, the adenovirus sequences employed are the cis-acting 5′ and 3′ inverted terminal repeat (ITR) sequences of an adenovirus (which function as origins of replication) and the native 5′ packaging/enhancer domain, that contains sequences necessary for packaging linear Ad genomes and enhancer elements for the E1 promoter. The adenovirus sequences may be modified to contain desired deletions, substitutions, or mutations, provided that the desired function is not eliminated.

The AAV sequences useful in the above chimeric vector are the viral sequences from which the rep and cap polypeptide encoding sequences are deleted. More specifically, the AAV sequences employed are the cis-acting 5′ and 3′ inverted terminal repeat (ITR) sequences. These chimeras are characterized by high titer transgene delivery to a host cell and the ability to stably integrate the transgene into the host cell chromosome (U.S. Pat. No. 5,871,983, specifically incorporate herein by reference). In the hybrid vector construct, the AAV sequences are flanked by the selected adenovirus sequences discussed above. The 5′ and 3′ AAV ITR sequences themselves flank a selected transgene sequence and associated regulatory elements, described below. Thus, the sequence formed by the transgene and flanking 5′ and 3′ AAV sequences may be inserted at any deletion site in the adenovirus sequences of the vector. For example, the AAV sequences are desirably inserted at the site of the deleted E1a/E1b genes of the adenovirus. Alternatively, the AAV sequences may be inserted at an E3 deletion, E2a deletion, and so on. If only the adenovirus 5′ ITR/packaging sequences and 3′ ITR sequences are used in the hybrid virus, the AAV sequences are inserted between them.

The transgene sequence of the vector and recombinant virus can be a gene, a nucleic acid sequence or reverse transcript thereof, heterologous to the adenovirus sequence, which encodes a protein, polypeptide or peptide fragment of interest. The transgene is operatively linked to regulatory components in a manner which permits transgene transcription. The composition of the transgene sequence will depend upon the use to which the resulting hybrid vector will be put. For example, one type of transgene sequence includes a therapeutic gene which expresses a desired gene product in a host cell. These therapeutic genes or nucleic acid sequences typically encode products for administration and expression in a patient in vivo or ex vivo to replace or correct an inherited or non-inherited genetic defect or treat an epigenetic disorder or disease.

I. Non-Viral Transformation

Suitable methods for nucleic acid delivery for transformation of an organelle, a cell, a tissue or an organism for use with the current disclosure are believed to include virtually any method by which a nucleic acid (e.g., DNA) can be introduced into an organelle, a cell, a tissue or an organism, as described herein or as would be known to one of ordinary skill in the art. Such methods include, but are not limited to, direct delivery of DNA such as by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harland and Weintraub, 1985; U.S. Pat. No. 5,789,215, incorporated herein by reference); by electroporation (U.S. Pat. No. 5,384,253, incorporated herein by reference); by calcium phosphate precipitation (Graham and Van Der Eb, 1973; Chen and Okayama, 1987; Rippe et al., 1990); by using DEAE-dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991); by microprojectile bombardment (PCT Application Nos. WO 94/09699 and 95/06128; U.S. Pat. Nos. 5,610,042; 5,322,783, 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); or by PEG-mediated transformation of protoplasts (Omirulleh et al., 1993; U.S. Pat. Nos. 4,684,611 and 4,952,500, each incorporated herein by reference); by desiccation/inhibition-mediated DNA uptake (Potrykus et al., 1985). Through the application of techniques such as these, organelle(s), cell(s), tissue(s) or organism(s) may be stably or transiently transformed.

Injection.

In certain embodiments, a nucleic acid may be delivered to an organelle, a cell, a tissue or an organism via one or more injections (i.e., a needle injection), such as, for example, either subcutaneously, intradermally, intramuscularly, intervenously or intraperitoneally. Methods of injection of vaccines are well known to those of ordinary skill in the art (e.g., injection of a composition comprising a saline solution). Further embodiments of the present disclosure include the introduction of a nucleic acid by direct microinjection. Direct microinjection has been used to introduce nucleic acid constructs into Xenopus oocytes (Harland and Weintraub, 1985).

Electroporation.

In certain embodiments of the present disclosure, a nucleic acid is introduced into an organelle, a cell, a tissue or an organism via electroporation. Electroporation involves the exposure of a suspension of cells and DNA to a high-voltage electric discharge. In some variants of this method, certain cell wall-degrading enzymes, such as pectin-degrading enzymes, are employed to render the target recipient cells more susceptible to transformation by electroporation than untreated cells (U.S. Pat. No. 5,384,253, incorporated herein by reference). Alternatively, recipient cells can be made more susceptible to transformation by mechanical wounding.

Transfection of eukaryotic cells using electroporation has been quite successful. Mouse pre-B lymphocytes have been transfected with human κ-immunoglobulin genes (Potter et al., 1984), and rat hepatocytes have been transfected with the chloramphenicol acetyltransferase gene (Tur-Kaspa et al., 1986) in this manner.

To effect transformation by electroporation in cells such as, for example, plant cells, one may employ either friable tissues, such as a suspension culture of cells or embryogenic callus or alternatively one may transform immature embryos or other organized tissue directly. In this technique, one would partially degrade the cell walls of the chosen cells by exposing them to pectin-degrading enzymes (pectolyases) or mechanically wounding in a controlled manner. Examples of some species which have been transformed by electroporation of intact cells include maize (U.S. Pat. No. 5,384,253; Rhodes et al., 1995; D'Halluin et al., 1992), wheat (Zhou et al., 1993), tomato (Hou and Lin, 1996), soybean (Christou et al., 1987) and tobacco (Lee et al., 1989).

One also may employ protoplasts for electroporation transformation of plant cells (Bates, 1994; Lazzeri, 1995). For example, the generation of transgenic soybean plants by electroporation of cotyledon-derived protoplasts is described by Dhir and Widholm in International Patent Application No. WO 92/17598, incorporated herein by reference. Other examples of species for which protoplast transformation has been described include barley (Lazerri, 1995), sorghum (Battraw et al., 1991), maize (Bhattacharjee et al., 1997), wheat (He et al., 1994) and tomato (Tsukada, 1989).

Calcium Phosphate.

In other embodiments of the present disclosure, a nucleic acid is introduced to the cells using calcium phosphate precipitation. Human KB cells have been transfected with adenovirus 5 DNA (Graham and Van Der Eb, 1973) using this technique. Also in this manner, mouse L(A9), mouse C127, CHO, CV-1, BHK, NIH3T3 and HeLa cells were transfected with a neomycin marker gene (Chen and Okayama, 1987), and rat hepatocytes were transfected with a variety of marker genes (Rippe et al., 1990).

DEAE-Dextran: In another embodiment, a nucleic acid is delivered into a cell using DEAE-dextran followed by polyethylene glycol. In this manner, reporter plasmids were introduced into mouse myeloma and erythroleukemia cells (Gopal, 1985).

Sonication Loading.

Additional embodiments of the present disclosure include the introduction of a nucleic acid by direct sonic loading. LTK⁻ fibroblasts have been transfected with the thymidine kinase gene by sonication loading (Fechheimer et al., 1987). Liposome-Mediated Transfection. In a further embodiment of the disclosure, a nucleic acid may be entrapped in a lipid complex such as, for example, a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated is an nucleic acid complexed with Lipofectamine (Gibco BRL) or Superfect (Qiagen).

Liposome-mediated nucleic acid delivery and expression of foreign DNA in vitro has been very successful (Nicolau and Sene, 1982; Fraley et al., 1979; Nicolau et al., 1987). The feasibility of liposome-mediated delivery and expression of foreign DNA in cultured chick embryo, HeLa and hepatoma cells has also been demonstrated (Wong et al., 1980).

In certain embodiments of the disclosure, a liposome may be complexed with a hemagglutinating virus (HVJ). This has been shown to facilitate fusion with the cell membrane and promote cell entry of liposome-encapsulated DNA (Kaneda et al., 1989). In other embodiments, a liposome may be complexed or employed in conjunction with nuclear non-histone chromosomal proteins (HMG-1) (Kato et al., 1991). In yet further embodiments, a liposome may be complexed or employed in conjunction with both HVJ and HMG-1. In other embodiments, a delivery vehicle may comprise a ligand and a liposome.

Receptor-Mediated Transfection.

Still further, a nucleic acid may be delivered to a target cell via receptor-mediated delivery vehicles. These take advantage of the selective uptake of macromolecules by receptor-mediated endocytosis that will be occurring in a target cell. In view of the cell type-specific distribution of various receptors, this delivery method adds another degree of specificity to the present disclosure.

Certain receptor-mediated gene targeting vehicles comprise a cell receptor-specific ligand and a nucleic acid-binding agent. Others comprise a cell receptor-specific ligand to which the nucleic acid to be delivered has been operatively attached. Several ligands have been used for receptor-mediated gene transfer (Wu and Wu, 1987; Wagner et al., 1990; Perales et al., 1994; Myers, EPO 0273085), which establishes the operability of the technique. Specific delivery in the context of another mammalian cell type has been described (Wu and Wu, 1993; incorporated herein by reference). In certain aspects of the present disclosure, a ligand will be chosen to correspond to a receptor specifically expressed on the target cell population.

In other embodiments, a nucleic acid delivery vehicle component of a cell-specific nucleic acid targeting vehicle may comprise a specific binding ligand in combination with a liposome. The nucleic acid(s) to be delivered are housed within the liposome and the specific binding ligand is functionally incorporated into the liposome membrane. The liposome will thus specifically bind to the receptor(s) of a target cell and deliver the contents to a cell. Such systems have been shown to be functional using systems in which, for example, epidermal growth factor (EGF) is used in the receptor-mediated delivery of a nucleic acid to cells that exhibit upregulation of the EGF receptor.

In still further embodiments, the nucleic acid delivery vehicle component of a targeted delivery vehicle may be a liposome itself, which will preferably comprise one or more lipids or glycoproteins that direct cell-specific binding. For example, lactosyl-ceramide, a galactose-terminal asialganglioside, have been incorporated into liposomes and observed an increase in the uptake of the insulin gene by hepatocytes (Nicolau et al., 1987). It is contemplated that the tissue-specific transforming constructs of the present disclosure can be specifically delivered into a target cell in a similar manner.

J. Expression Systems

Numerous expression systems exist that comprise at least a part or all of the compositions discussed above. Prokaryote- and/or eukaryote-based systems can be employed for use with the present disclosure to produce nucleic acid sequences, or their cognate polypeptides, proteins and peptides. Many such systems are commercially and widely available.

The insect cell/baculovirus system can produce a high level of protein expression of a heterologous nucleic acid segment, such as described in U.S. Pat. Nos. 5,871,986 and 4,879,236, both herein incorporated by reference, and which can be bought, for example, under the name MaxBac® 2.0 from Invitrogen® and BacPack™ Baculovirus Expression System From Clontech®.

Other examples of expression systems include Stratagene®'s Complete Control™ Inducible Mammalian Expression System, which involves a synthetic ecdysone-inducible receptor, or its pET Expression System, an E. coli expression system. Another example of an inducible expression system is available from Invitrogen®, which carries the T-Rex™ (tetracycline-regulated expression) System, an inducible mammalian expression system that uses the full-length CMV promoter. Invitrogen® also provides a yeast expression system called the Pichia methanolica Expression System, which is designed for high-level production of recombinant proteins in the methylotrophic yeast Pichia methanolica. One of skill in the art would know how to express a vector, such as an expression construct, to produce a nucleic acid sequence or its cognate polypeptide, protein, or peptide.

Primary mammalian cell cultures may be prepared in various ways. In order for the cells to be kept viable while in vitro and in contact with the expression construct, it is necessary to ensure that the cells maintain contact with the correct ratio of oxygen and carbon dioxide and nutrients but are protected from microbial contamination. Cell culture techniques are well documented.

One embodiment of the foregoing involves the use of gene transfer to immortalize cells for the production of proteins. The gene for the protein of interest may be transferred as described above into appropriate host cells followed by culture of cells under the appropriate conditions. The gene for virtually any polypeptide may be employed in this manner. The generation of recombinant expression vectors, and the elements included therein, are discussed above. Alternatively, the protein to be produced may be an endogenous protein normally synthesized by the cell in question.

Examples of useful mammalian host cell lines are Vero and HeLa cells and cell lines of Chinese hamster ovary, W138, BHK, COS-7, 293, HepG2, NIH3T3, RIN and MDCK cells. In addition, a host cell strain may be chosen that modulates the expression of the inserted sequences, or modifies and process the gene product in the manner desired. Such modifications (e.g., glycosylation) and processing (e.g., cleavage) of protein products may be important for the function of the protein. Different host cells have characteristic and specific mechanisms for the post-translational processing and modification of proteins. Appropriate cell lines or host systems can be chosen to insure the correct modification and processing of the foreign protein expressed.

A number of selection systems may be used including, but not limited to, HSV thymidine kinase, hypoxanthine-guanine phosphoribosyltransferase and adenine phosphoribosyltransferase genes, in tk-, hgprt- or aprt-cells, respectively. Also, anti-metabolite resistance can be used as the basis of selection for dhfr, that confers resistance to; gpt, that confers resistance to mycophenolic acid; neo, that confers resistance to the aminoglycoside G418; and hygro, that confers resistance to hygromycin.

### III. CELL-BASED CANCER VACCINES

In one aspect, the present disclosure addresses cancer therapy. Cancer cells may be obtained from virtually any source, and may be primary (obtained directly from a patient without significant culturing), passaged cancer cells, or cancer cell lines. The appropriate cancer cell will be engineered to express Ig Fc on its cell surface. Following (re)introduction into a patient, the patient's own immune system will activate CD8+ T cells. Optionally, these vaccines may be provided with one or more immunomodulatory agents, including adjuvants, cytokines and TLR3/RIG-I ligands.

An appropriate cancer cell can be a breast cancer cell, lung cancer cell, colon cancer cell, pancreatic cancer cell, renal cancer cell, stomach cancer cell, liver cancer cell, bone cancer cell, hematological cancer cell (e.g., leukemia or lymphoma), neural tissue cancer cell, melanoma cell, ovarian cancer cell, testicular cancer cell, prostate cancer cell, cervical cancer cell, vaginal cancer cell, or bladder cancer cell. The cancer may be primary, metastatic, recurrent and/or multi-drug resistant. The cancer cell may be taken from and returned to the same patient following introduction of the machinery to express Fc on the cell's surface (autologous ex vivo therapy), or the cell may be “foreign” or “heterologous” to the patient, but nonetheless be of a type sufficiently similar to the cancer in the patient such that a therapeutic effect will be elicited. In the latter situation, the method may involve determining the cancer type in the patient to assess the nature of the engineered cancer cell that may prove most efficacious.

Pharmaceutical compositions comprising engineered cancer cells are provided herein. In general, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Other suitable pharmaceutical excipients include starch, glucose, lactose, sucrose, saline, dextrose, gelatin, malt, rice, flour, chalk, silica gel, sodium stearate, glycerol monostearate, talc, sodium chloride, dried skim milk, glycerol, propylene glycol, water, ethanol and the like. In the case of a vaccine, adjuvants and immunomodulatory compounds also are contemplated.

The cell of the present disclosure may include classic pharmaceutical preparations formulated for various routes of administration. Administration of these compositions will be via any common route, including oral, nasal, buccal, rectal, dermal, vaginal or topical. Alternatively, administration may be by intradermal, subcutaneous, intramuscular, intraperitoneal or intravenous injection. Such compositions would normally be administered as pharmaceutically acceptable compositions, described supra. Of particular interest is direct intratumoral administration, perfusion of a tumor, or administration local or regional to a tumor, for example, in the local or regional vasculature or lymphatic system, or in a resected tumor bed.

The vaccines may contain live cells or non-live cells. The non-live cells may have been frozen or fixed. Solutions may include pharmacologically acceptable salts, and may be lyophilized and prepared in sterile buffer or water for administration. Under ordinary conditions of storage and use, these preparations contain a preservative to prevent the growth of microorganisms.

### IV. CANCER THERAPY

A. Cancers

Cancer results from the outgrowth of a clonal population of cells from tissue. The development of cancer, referred to as carcinogenesis, can be modeled and characterized in a number of ways. An association between the development of cancer and inflammation has long-been appreciated. The inflammatory response is involved in the host defense against microbial infection, and also drives tissue repair and regeneration. Considerable evidence points to a connection between inflammation and a risk of developing cancer, i.e., chronic inflammation can lead to dysplasia.

Cancer cells to which the methods of the present disclosure can be applied include generally any cell. An appropriate cancer target can be a breast cancer, lung cancer, colon cancer, pancreatic cancer, renal cancer, stomach cancer, liver cancer, bone cancer, hematological cancer (e.g., leukemia or lymphoma), neural tissue cancer, melanoma, ovarian cancer, testicular cancer, prostate cancer, brain cancer, cervical cancer, vaginal cancer, or bladder cancer cell. In addition, the methods of the disclosure can be applied to a wide range of species, e.g., humans, non-human primates (e.g., monkeys, baboons, or chimpanzees), horses, cattle, pigs, sheep, goats, dogs, cats, rabbits, guinea pigs, gerbils, hamsters, rats, and mice. Cancers may also be recurrent, metastatic and/or multi-drug resistant, and the methods of the present disclosure may be particularly applied to such cancers so as to render them resectable, to prolong or re-induce remission, to inhibit angiogenesis, to prevent or limit metastasis, and/or to treat multi-drug resistant cancers. At a cellular level, this may translate into killing cancer cells, inhibiting cancer cell growth, or otherwise reversing or reducing the malignant phenotype of tumor cells.

B. Combination Therapies

In the context of the present disclosure, it also is contemplated that engineered cancer cells described herein could be used similarly in conjunction with chemo- or radiotherapeutic intervention, or other treatments. It also may prove effective, in particular, to combine these vaccines with other therapies that target different aspects of the immune response.

To kill cells, inhibit cell growth, inhibit metastasis, inhibit angiogenesis or otherwise reverse or reduce the malignant phenotype of tumor cells, using the methods and compositions of the present disclosure, one may administer an engineered cell according to the present disclosure and at least one other agent. These compositions would be provided in a combined amount effective to kill or inhibit proliferation of the cell. This process may involve contacting the cells and the other agent(s) or factor(s) at the same time. This may be achieved by contacting the patient with a single composition or pharmacological formulation that includes both agents, or by contacting the patient with two distinct compositions or formulations, at the same time, wherein one composition includes the engineered cell according to the present disclosure and the other includes the other agent.

Alternatively, the engineered cell may precede or follow the other agent treatment by intervals ranging from minutes to weeks. In embodiments where the other agent and the engineered cell are applied separately to the patient, one would generally ensure that a significant period of time did not expire between the time of each delivery, such that the agent and engineered cell would still be able to exert an advantageously combined effect on the cell. In such instances, it is contemplated that one would contact the cell with both modalities within about 12-24 hours of each other and, more preferably, within about 6-12 hours of each other, with a delay time of only about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It also is conceivable that more than one administration of either engineered cell or the other agent will be desired. Various combinations may be employed, where the engineered cell according to the present disclosure is “A” and the other therapy is “B”, as exemplified below:

| | | | | | |
|---|---|---|---|---|---|
| A/B/A | B/A/B | B/B/A | A/A/B | B/A/A | A/B/B |
| B/B/B/A | B/B/A/B | A/A/B/B | A/B/A/B | A/B/B/A | B/B/A/A |
| B/A/B/A | B/A/A/B | B/B/B/A | A/A/A/B | B/A/A/A | A/B/A/A |
| A/A/B/A | A/B/B/B | B/A/B/B | B/B/A/B | | |

Other combinations are contemplated. Again, to achieve cell killing, both agents are delivered to a cell in a combined amount effective to kill the cell.

1. Chemotherapy

The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis. Most chemotherapeutic agents fall into the following categories: alkylating agents, antimetabolites, antitumor antibiotics, mitotic inhibitors, and nitrosoureas.

Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gamma I and calicheamicin omega I; dynemicin, including dynemicin A, uncialamycin, and derivatives thereof; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel and doxetaxel; chlorambucil; gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids such as retinoic acid; capecitabine; cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, paclitaxel, docetaxel, gemcitabien, navelbine, farnesyl-protein tansferase inhibitors, transplatinum, 5-fluorouracil, vincristin, vinblastin and methotrexate and pharmaceutically acceptable salts, acids or derivatives of any of the above.

In particular, tyrosine kinase inhibitors are a class of agent that can be used in combination with the compounds of the present application. For example, Imatinib is a tyrosine-kinase inhibitor used in the treatment of multiple cancers, most notably Philadelphia chromosome-positive (Ph⁺) chronic myelogenous leukemia (CML). Like all tyrosine-kinase inhibitors, imatinib works by preventing a tyrosine kinase enzyme, in this case BCR-Abl, from phosphorylating subsequent proteins and initiating the signaling cascade necessary for cancer development, thus preventing the growth of cancer cells and leading to their death by apoptosis. Because the BCR-Abl tyrosine kinase enzyme exists only in cancer cells and not in healthy cells, imatinib works as a form of targeted therapy—only cancer cells are killed through the drug's action. In this regard, imatinib was one of the first cancer therapies to show the potential for such targeted action, and is often cited as a paradigm for research in cancer therapeutics.

Various classes of chemotherapeutic agents are comtemplated for use with the present disclosure. For example, selective estrogen receptor antagonists (“SERMs”), such as Tamoxifen, 4-hydroxy Tamoxifen (Afimoxfene), Falsodex, Raloxifene, Bazedoxifene, Clomifene, Femarelle, Lasofoxifene, Ormeloxifene, and Toremifene.

Chemotherapeutic agents contemplated to be of use, include, e.g., camptothecin, actinomycin-D, mitomycin C. The disclosure also encompasses the use of a combination of one or more DNA damaging agents, whether radiation-based or actual compounds, such as the use of X-rays with cisplatin or the use of cisplatin with etoposide. The agent may be prepared and used as a combined therapeutic composition, or kit, by combining it with a MUC1 peptide, as described above.

Heat shock protein 90 is a regulatory protein found in many eukaryotic cells. HSP90 inhibitors have been shown to be useful in the treatment of cancer. Such inhibitors include Geldanamycin, 17-(Allylamino)-17-demethoxygeldanamycin, PU-H71 and Rifabutin.

Agents that directly cross-link DNA or form adducts are also envisaged. Agents such as cisplatin, and other DNA alkylating agents may be used. Cisplatin has been widely used to treat cancer, with efficacious doses used in clinical applications of 20 mg/m² for 5 days every three weeks for a total of three courses. Cisplatin is not absorbed orally and must therefore be delivered via injection intravenously, subcutaneously, intratumorally or intraperitoneally.

Agents that damage DNA also include compounds that interfere with DNA replication, mitosis and chromosomal segregation. Such chemotherapeutic compounds include adriamycin, also known as doxorubicin, etoposide, verapamil, podophyllotoxin, and the like. Widely used in a clinical setting for the treatment of neoplasms, these compounds are administered through bolus injections intravenously at doses ranging from 25-75 mg/m² at 21 day intervals for doxorubicin, to 35-50 mg/m² for etoposide intravenously or double the intravenous dose orally. Microtubule inhibitors, such as taxanes, also are contemplated. These molecules are diterpenes produced by the plants of the genus Taxus, and include paclitaxel and docetaxel.

2. Radiotherapy

Radiotherapy, also called radiation therapy, is the treatment of cancer and other diseases with ionizing radiation. Ionizing radiation deposits energy that injures or destroys cells in the area being treated by damaging their genetic material, making it impossible for these cells to continue to grow. Although radiation damages both cancer cells and normal cells, the latter are able to repair themselves and function properly.

Radiation therapy used according to the present disclosure may include, but is not limited to, the use of γ-rays, X-rays, and/or the directed delivery of radioisotopes to tumor cells. Other forms of DNA damaging factors are also contemplated such as microwaves and UV-irradiation. It is most likely that all of these factors induce a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

Radiotherapy may comprise the use of radiolabeled antibodies to deliver doses of radiation directly to the cancer site (radioimmunotherapy). Antibodies are highly specific proteins that are made by the body in response to the presence of antigens (substances recognized as foreign by the immune system). Some tumor cells contain specific antigens that trigger the production of tumor-specific antibodies. Large quantities of these antibodies can be made in the laboratory and attached to radioactive substances (a process known as radiolabeling). Once injected into the body, the antibodies actively seek out the cancer cells, which are destroyed by the cell-killing (cytotoxic) action of the radiation. This approach can minimize the risk of radiation damage to healthy cells.

Conformal radiotherapy uses the same radiotherapy machine, a linear accelerator, as the normal radiotherapy treatment but metal blocks are placed in the path of the x-ray beam to alter its shape to match that of the cancer. This ensures that a higher radiation dose is given to the tumor. Healthy surrounding cells and nearby structures receive a lower dose of radiation, so the possibility of side effects is reduced. A device called a multi-leaf collimator has been developed and can be used as an alternative to the metal blocks. The multi-leaf collimator consists of a number of metal sheets which are fixed to the linear accelerator. Each layer can be adjusted so that the radiotherapy beams can be shaped to the treatment area without the need for metal blocks. Precise positioning of the radiotherapy machine is very important for conformal radiotherapy treatment and a special scanning machine may be used to check the position of internal organs at the beginning of each treatment.

High-resolution intensity modulated radiotherapy also uses a multi-leaf collimator. During this treatment the layers of the multi-leaf collimator are moved while the treatment is being given. This method is likely to achieve even more precise shaping of the treatment beams and allows the dose of radiotherapy to be constant over the whole treatment area.

Although research studies have shown that conformal radiotherapy and intensity modulated radiotherapy may reduce the side effects of radiotherapy treatment, it is possible that shaping the treatment area so precisely could stop microscopic cancer cells just outside the treatment area being destroyed. This means that the risk of the cancer coming back in the future may be higher with these specialized radiotherapy techniques.

Scientists also are looking for ways to increase the effectiveness of radiation therapy. Two types of investigational drugs are being studied for their effect on cells undergoing radiation. Radiosensitizers make the tumor cells more likely to be damaged, and radioprotectors protect normal tissues from the effects of radiation. Hyperthermia, the use of heat, is also being studied for its effectiveness in sensitizing tissue to radiation.

3. Immunotherapy

In the context of cancer treatment, immunotherapeutics, generally, rely on the use of immune effector cells and molecules to target and destroy cancer cells. Trastuzumab (Herceptin™) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells. The combination of therapeutic modalities, i.e., direct cytotoxic activity and inhibition or reduction of ErbB2 would provide therapeutic benefit in the treatment of ErbB2 overexpressing cancers.

In one aspect of immunotherapy, the tumor cell must bear some marker that is amenable to targeting, i.e., is not present on the majority of other cells. Many tumor markers exist and any of these may be suitable for targeting in the context of the present disclosure. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155. An alternative aspect of immunotherapy is to combine anticancer effects with immune stimulatory effects. Immune stimulating molecules also exist including: cytokines such as IL-2, IL-4, IL-12, GM-CSF, γ-IFN, chemokines such as MIP-1, MCP-1, IL-8 and growth factors such as FLT3 ligand. Combining immune stimulating molecules, either as proteins or using gene delivery in combination with a tumor suppressor has been shown to enhance anti-tumor effects (Ju et al., 2000). Moreover, antibodies against any of these compounds can be used to target the anti-cancer agents discussed herein.

Examples of immunotherapies currently under investigation or in use are immune adjuvants e.g., Mycobacterium bovis, Plasmodium falciparum, dinitrochlorobenzene and aromatic compounds (U.S. Pat. Nos. 5,801,005 and 5,739,169; Hui and Hashimoto, 1998; Christodoulides et al., 1998), cytokine therapy, e.g., interferons α, β, and γ; IL-1, GM-CSF and TNF (Bukowski et al., 1998; Davidson et al., 1998; Hellstrand et al., 1998) gene therapy, e.g., TNF, IL-1, IL-2, p53 (Qin et al., 1998; Austin-Ward and Villaseca, 1998; U.S. Pat. Nos. 5,830,880 and 5,846,945) and monoclonal antibodies, e.g., anti-ganglioside GM2, anti-HER-2, anti-p185 (Pietras et al., 1998; Hanibuchi et al., 1998; U.S. Pat. No. 5,824,311). It is contemplated that one or more anti-cancer therapies may be employed with the gene silencing therapies described herein.

Of particular interest are a ligand for TLR3 or RIG-I, including specifically poly I: C.

4. Surgery

Approximately 60% of persons with cancer will undergo surgery of some type, which includes preventative, diagnostic or staging, curative, and palliative surgery. Curative surgery is a cancer treatment that may be used in conjunction with other therapies, such as the treatment of the present disclosure, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and microscopically controlled surgery (Mohs' surgery). It is further contemplated that the present disclosure may be used in conjunction with removal of superficial cancers, precancers, or incidental amounts of normal tissue.

Upon excision of part or all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

5. Other Agents

It is contemplated that other agents may be used with the present disclosure. These additional agents include immunomodulatory agents, agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Immunomodulatory agents include tumor necrosis factor; interferon alpha, beta, and gamma; IL-2 and other cytokines; F42K and other cytokine analogs; or MIP-1, MCP-1, RANTES, and other chemokines. It is further contemplated that the upregulation of cell surface receptors or their ligands such as Fas/Fas ligand, DR4 or DR5/TRAIL (Apo-2 ligand) would potentiate the apoptotic inducing abilities of the present disclosure by establishment of an autocrine or paracrine effect on hyperproliferative cells. Increases intercellular signaling by elevating the number of GAP junctions would increase the anti-hyperproliferative effects on the neighboring hyperproliferative cell population. In other embodiments, cytostatic or differentiation agents can be used in combination with the present disclosure to improve the anti-hyerproliferative efficacy of the treatments. Inhibitors of cell adhesion are contemplated to improve the efficacy of the present disclosure. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with the present disclosure to improve the treatment efficacy.

There have been many advances in the therapy of cancer following the introduction of cytotoxic chemotherapeutic drugs. However, one of the consequences of chemotherapy is the development/acquisition of drug-resistant phenotypes and the development of multiple drug resistance. The development of drug resistance remains a major obstacle in the treatment of such tumors and therefore, there is an obvious need for alternative approaches such as gene therapy.

Another form of therapy for use in conjunction with chemotherapy, radiation therapy or biological therapy includes hyperthermia, which is a procedure in which a patient's tissue is exposed to high temperatures (up to 106° F.). External or internal heating devices may be involved in the application of local, regional, or whole-body hyperthermia. Local hyperthermia involves the application of heat to a small area, such as a tumor. Heat may be generated externally with high-frequency waves targeting a tumor from a device outside the body. Internal heat may involve a sterile probe, including thin, heated wires or hollow tubes filled with warm water, implanted microwave antennae, or radiofrequency electrodes.

A patient's organ or a limb is heated for regional therapy, which is accomplished using devices that produce high energy, such as magnets. Alternatively, some of the patient's blood may be removed and heated before being perfused into an area that will be internally heated. Whole-body heating may also be implemented in cases where cancer has spread throughout the body. Warm-water blankets, hot wax, inductive coils, and thermal chambers may be used for this purpose.

The skilled artisan is directed to “Remington's Pharmaceutical Sciences” 15th Edition, chapter 33, in particular pages 624-652. Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

It also should be pointed out that any of the foregoing therapies may prove useful by themselves in treating cancer.

### V. EXAMPLES

It will be understood that particular embodiments described herein are shown by way of illustration and not as limitations of the disclosure. The principal features of this disclosure can be employed in various embodiments without departing from the scope of the disclosure. All of the compositions and/or methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and/or methods and in the steps or in the sequence of steps of the method described herein without departing from the concept, spirit and scope of the disclosure. All such similar substitutes and modifications apparent to those skilled in the art are deemed to be within the spirit, scope and concept of the disclosure as defined by the appended claims.

### Example 1—Materials and Methods

Mice.

OT-I and OT-II mice were obtained from Jackson Laboratories (Bar Harbor, Me.) Control C57BL/6 mice were obtained from the UT Southwestern mouse breeding core facility. Mice were maintained in specific pathogen-free conditions. Mice were used between 6 and 12 wk of age.

Cell Lines and DCs.

EG7 cells (ATCC, Manassas, Va.) and murine primary cells were cultured in complete RPMI-1640 supplemented with 10% FCS, 100 U/ml penicillin, 100 μg/ml streptomycin, 2 mM L-glutamine, 10 mM HEPES, and 1 mM sodium pyruvate (all from Sigma). BMDCs were generated from bone marrow progenitors. Cells were harvested from femurs and iliac bones of WT mice, cultured for 5 days in complete RPMI-1640 supplemented with 5% FCS, 100 U/ml penicillin, 100 g/ml streptomycin, 2 mM L-glutamine, 10 mM HEPES, and 1 mM sodium pyruvate (all from Sigma) and GM-CSF. Media was replenished on day 2 and day 4 of culture. Splenic FLT3 ligand induced DCs were obtained as described previously (Pasare and Medzhitov, 2003).

Reagents and Antibodies.

Allophyocyanin (APC) labeled anti-CD11c, phycoerythrin (PE) labeled anti-Thy 1.2, anti-Mouse IgG1 Biotin and Streptavidin APC (all from Biolegend, San Diego, Calif.) were used for staining of cells for flow cytometry analysis.

Retroviral Transduction.

Retrovirus was prepared from 293T cells (ATCC, Manassas, Va.) transfected with MSCV 2.2, VSV-g (Clontech, Mountain View, Calif.) and pcl-ECO (Imgenex, San Diego, Calif.) and VSV-g expressing plasmids using PEI transfection reagent (Sigma). Virus was harvested from 293T cultures after 24 h of transfection and centrifuged with EG7 cells for 90 minutes at 24° C. at 1200 RPM. Transformed cells were grown as above in 10% FCS containing RPMI and repeatedly sorted for high expression of GFP using FACS on a MoFLo cell sorter (Beckman Coulter, Brea, Calif.).

Purification of T Cells.

Spleens and lymph nodes were harvested from 8- to 12-week-old mice. CD4+ and CD8+ T cells were purified from the spleens by negative selection as previously described (Pasare and Medzhitov, 2004).

T Cell Activation Assays.

BMDCs were prepared as above and cultured with modified EG7 for 12 hours. Cells were stained for CD11c APC and Thy 1.2 PE as above and sorted for positive expression of CD11c and the absence of Thy 1.2. Purified DCs were cultured with purified T cells from OT-I and OT-II animals as above at various ratios of DCs to T cells for 2 days at 37° C. in round bottom 96-well plates. Proliferation of T cells was determined by incorporation of (³H) thymidine for the last 12-16 hr of the culture (Perkin Elmer, Waltham, Mass.). For blocking experiments DCs were treated with 2.4G2 antibody (BD Biosciences, San Jose, Calif.) at a concentration of 1 ug/mL for 30 minutes prior to the incubation with tumor for ˜12 hours.

Intracellular Staining.

BMDCs were cultured (1:1) with EG7-Fc or EG7-EV for 12 hrs. Cells were stained for CD11c and Thy1.2 and sorted for CD11c positive and Thy1.2 negative population by FACS. Naive OT-I cells were cultured with purified CD11c positive BMDCs (1:5 ratio) for 2 days at 37° C. in 48 well plate. Primed OT-I cells were stimulated with 50 ng/ml phorbol myristate acetate (PMA) and 1 mM ionomycin in the presence of 1 mg/ml brefeldin A for 5 hr, followed by surface staining, fixed with 4% paraformaldehyde, permeabilized with 0.3% saponin, and stained for intracellular cytokines. The stained cells were analyzed with a FACSCalibur flow cytometer (BD Biosciences). Data were analyzed with FlowJo software (Tree Star).

T Cell Cytotixicity Assays.

Cytolytic assay. OT-I T cells were primed with EG7-EV or EG7-Fc fed BMDCs for 72 hours and used as effector cells. EG7-EV and EL4 cells mixed 1:1 were used as target cells and cultured with (5:1, 15:1 and 30:1 effector/target ratios) and without effector cells for 12 hours. The percentage of remaining EG7-EV cells (GFP+) were then measured by flow cytometry. Antigen specific cytolysis were calculated with the following formula: % Cytolysis=(% EG7_{effector}−% EG7_{no effector})/% EG7_{no effector}.

Tumor Implantation Experiments.

For engraftment studies, 5×10⁵ modified EG7 cells were implanted subcutaneously into the inguinal region of mice. Tumors were measured 2-3 times per week by caliper and mice with tumors greater than 2.5 cm in any one dimension were sacrificed. Tumors were also quantified by mass at the time of death. Tumor volume was calculated, as described before, using a standard formula for estimation of volume based on two dimensional caliper measurements (Euhus et al., 1986). For tumor vaccine experiments, modified EG7 cells were treated with 50 μg/ml of mitomycin C in PBS for 5 hours at 37° C. Cells were washed 4 times with 10% FCS in PBS, counted and 5×10⁵ treated cells were injected into mice. Draining lymph nodes were harvested from tumor bearing mice, purified by negative selection and allowed to proliferate on BMDCs fed tumor as above. Therapeutic vaccine experiments were carried out as above with 5×10⁵ cells used in initial tumor implantation and for vaccine dose. Measurements were performed by member of the lab (T.B.) who was blinded to therapy for the entire duration of the experiment.

Live Cell Imaging.

A pDV Deltavision deconvolution microscope equipped with a 20× Olympus objective, Cool Snap HQ2 camera, and FITC filters was used for all imaging experiments. The time-lapsed imaging was controlled with Deltavision SoftWoRx software. A single brightfield and fluorescent image was acquired every 10 s for 4 hours at 37° C. Images were processed and interaction times were analyzed in ImageJ (NIH). DC:tumor cell interactions that initiated and commenced within experiment duration were analyzed. Interaction was defined as when the DC showed membrane spreading across the cancer cell surface or membrane projections that continually sampled the cancer cell surface.

### Example 2—Results

Engineering IgG1 Fe-Tagged Tumor Cells.

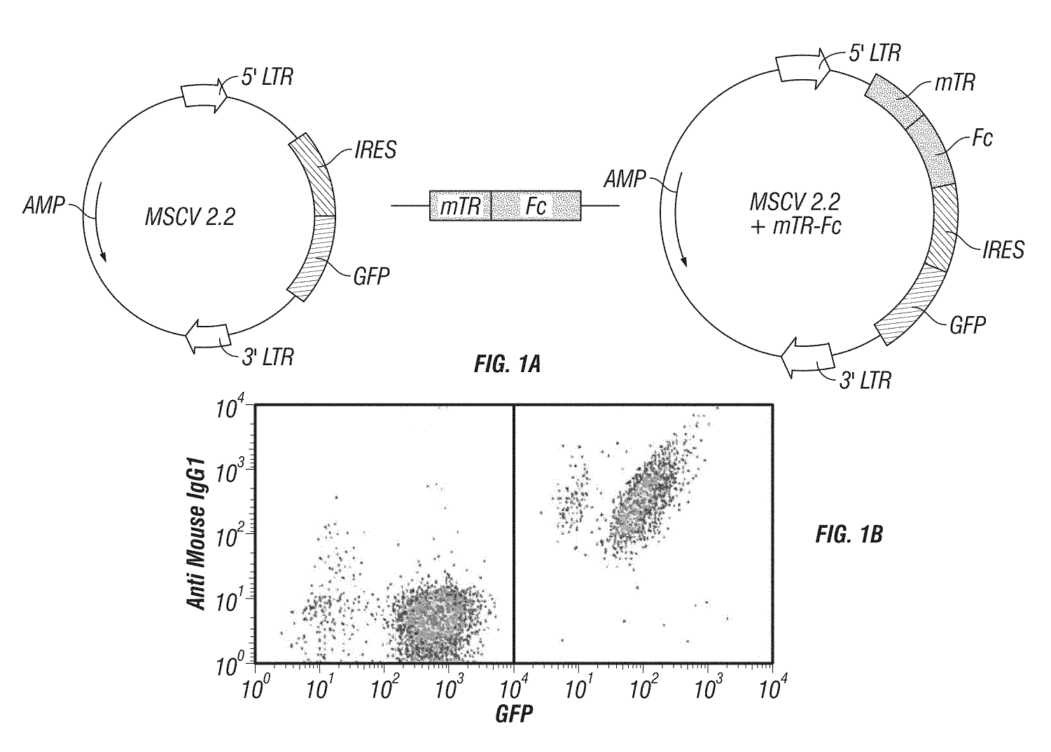

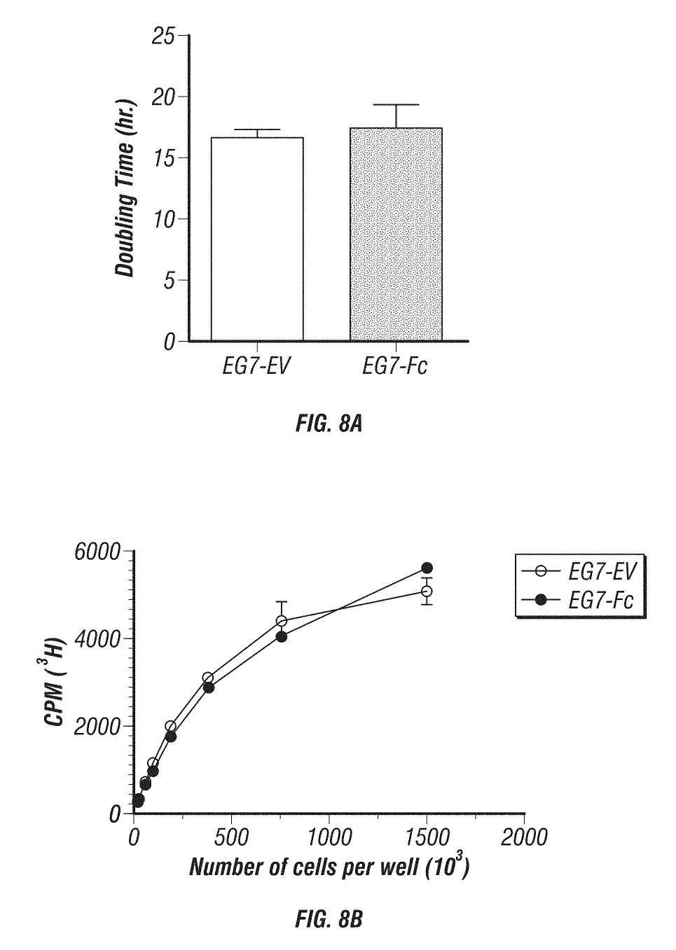

To direct trafficking of tumor cells and their antigens to Fc receptors on dendritic cells, the inventors expressed the Fc region of IgG1 on the surface of the tumor cell line EG7. The CH2 and CH3 domains (residues 237 to 430) of murine IgG1 Fc can be efficiently expressed on the cell surface in reverse orientation by fusing IgG1 Fc with the transmembrane domain of transferrin receptor (Stabila et al., 1998; Takashima et al., 2005). This chimeric protein approach was previously exploited to propagate pseudorabies virus with the Fc portion incorporated into its viral envelope. This modified virus was then used for immunization studies (Takashima et al., 2005). The inventors cloned the murine chimeric IgG1 Fc-transferrin fusion into a retroviral vector (MSCV 2.2) (FIG. 1A) and transduced EG7 cells (Moore et al., 1988) (the murine lymphoma cell line EL4 that expresses the model antigen ovalbumin). The resulting Fc-transferrin expressing EG7 cells are hereafter referred to as EG7-Fc. A control cell line was constructed by transducing EG7 cells with an MSCV vector lacking the IgG1 Fc insert (EG7-Empty vector, EG7-EV). Both EG7 and EL4 cells form aggressive tumors when injected subcutaneously in mice and ultimately result in lethality in 1-2 months. The pMIG vector MSCV 2.2 contains an IRES sequence followed by GFP. Thus, both transduced cell lines express GFP, but surface Fc expression was seen only in cells transfected with the IgG1-Fc containing vector (FIG. 1B). Polyclonal cultures of transduced cells were derived from sorted cells that expressed high levels of GFP. The inventors found no difference in the doubling time or ³H-thymidine incorporation rate in these two modified cell lines, suggesting that retroviral modification did not alter the growth kinetics of the transduced cells (FIGS. 8A-B).

IgG1-Fc Expressing Tumors Enhance Dendritic Cell Cross-Presentation.

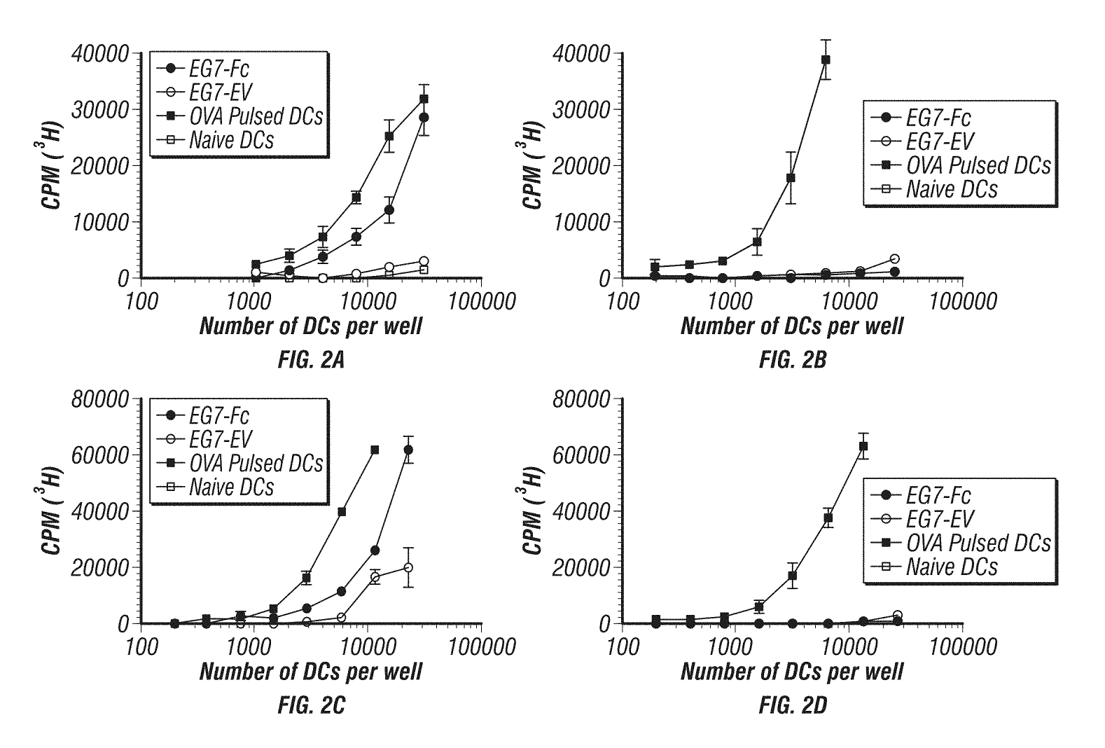

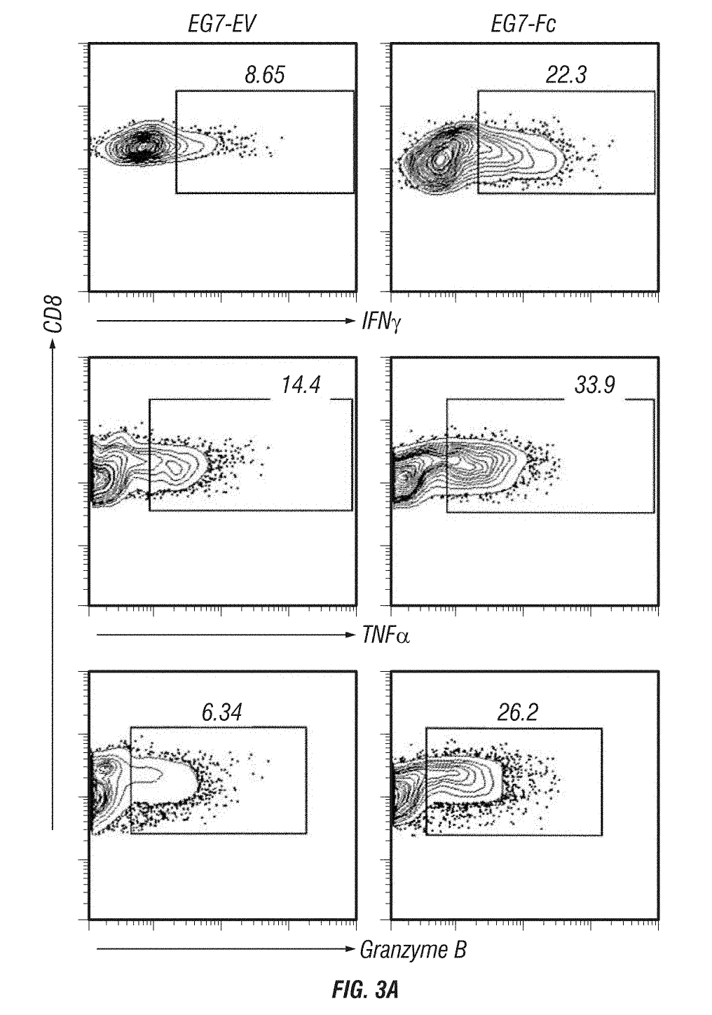

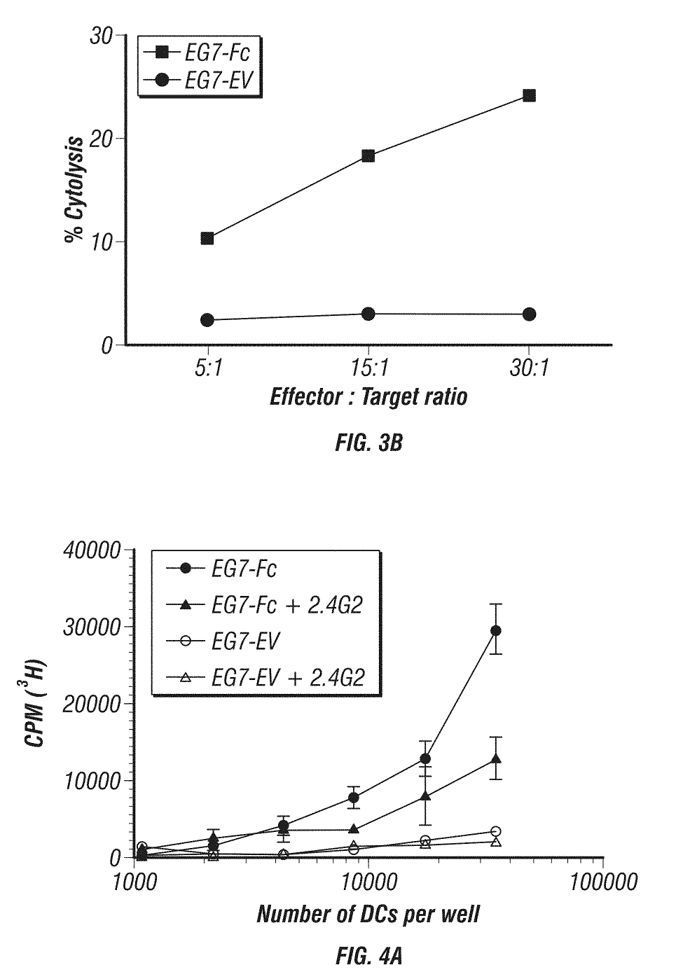

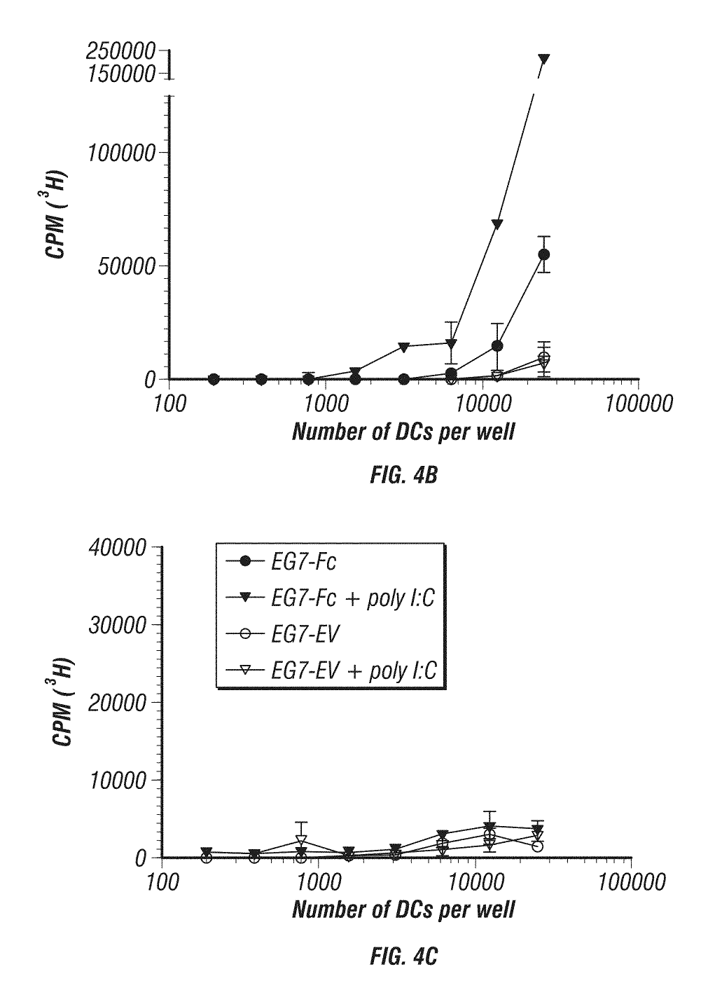

The inventors hypothesized that engagement of Fc receptors on dendritic cells by tumor cells that expressed IgG1-Fc would enhance processing of tumor-specific and tumor-associated antigens and their presentation to CD4 and CD8 T lymphocytes. To test this hypothesis, the inventors incubated bone marrow-derived dendritic cells (Mayordomo et al., 1995) (FIGS. 2A-B) or DCs derived from the spleen after Flt3L injection (Mach et al. 2000) (FIGS. 2C-D), with live EG7-Fc or EG7-EV for 12 hours. DCs were then purified to >99.9% purity via fluorescence activated cell sorting (FACS) and incubated with OVA-specific CD8 and CD4 T cells from OT-I and OT-II TCR transgenic (Tg) animals, respectively (Clarke et al., 2000; Barnden et al., 1998). CD8 T cells from OT-I TCR Tg mice co-cultured with DCs that were pre-incubated with EG7-Fc tumor cells showed significantly greater proliferation compared to OT-I T cells co-cultured with DCs pre-incubated with control EG7-EV cells (FIGS. 2A, 2C). Surprisingly, CD4 OT-II lymphocytes co-cultured with DCs pre-incubated with either cell line showed limited proliferation (FIGS. 2B, 2D). The inventors also found that DCs that were pre-incubated with EG7-Fc tumor cells induced greater IFN-γ, TNF-α and Granzyme B production by CD8 T cells, and these cells were able to kill target cells more efficiently than EG7-EV induced CD8+ cells (FIGS. 3A-B). Importantly, incubation of DCs with the FcγRII and FcγRIII blocking antibody 2.4G2 prior to co-culture with tumor cell lines was sufficient to blunt the enhancement of CD8 priming seen after incubation with EG7-Fc tumor cells (FIG. 4A).

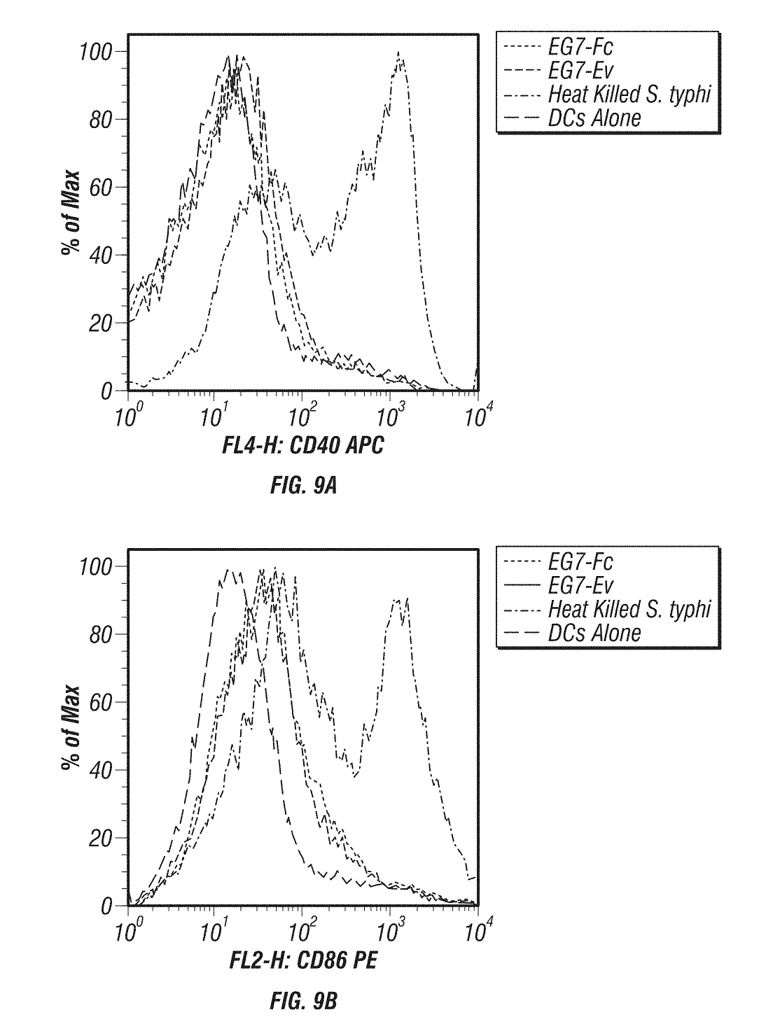

The observation that targeting of tumors to Fc receptors enhances the priming of CD8, but not CD4 T cells, argues that the expression of the Fc portion of IgG1 on tumor cells enhances cross-presentation of tumor cell-derived antigens, but does not enhance presentation of tumor-derived antigens by MHC class-II. Notably, no significant differences were observed in the cell surface expression of the DC activation molecules CD86 and CD40 after 12 hours of culture with tumor cells (FIGS. 9A-B), suggesting that the enhanced cross-priming the inventors observed was unlikely to be dependent on these costimulatory molecules. Therefore, the inventors next tested whether they could further enhance T cell responses to Fc-targeted tumor antigens by activating DCs exposed to EG7-Fc using TLR ligands. Since the TLR3 ligand poly I:C is approved for use in humans and has been shown to be an effective adjuvant in vivo (Longhi et al., 2009), the inventors decided to test its ability to influence CD8 T cell responses induced by DCs incubated with tumor cells. Stimulation of DCs with poly I:C greatly enhanced cross-presentation of EG7-Fc derived antigens to CD8 T cells (FIGS. 4B, 4C). In contrast, addition of poly I:C did not enhance CD4 T cell priming under similar conditions. Taken together, these findings suggest that effective cross-presentation and CD8 T cell activation can be induced by simply targeting tumor cargo to Fc receptors on DCs, and that concomitant activation of TLR3 can further enhance these CD8 T cell responses.

IgG1-Fc Expressing Tumors Interact Longer with BMDCs.

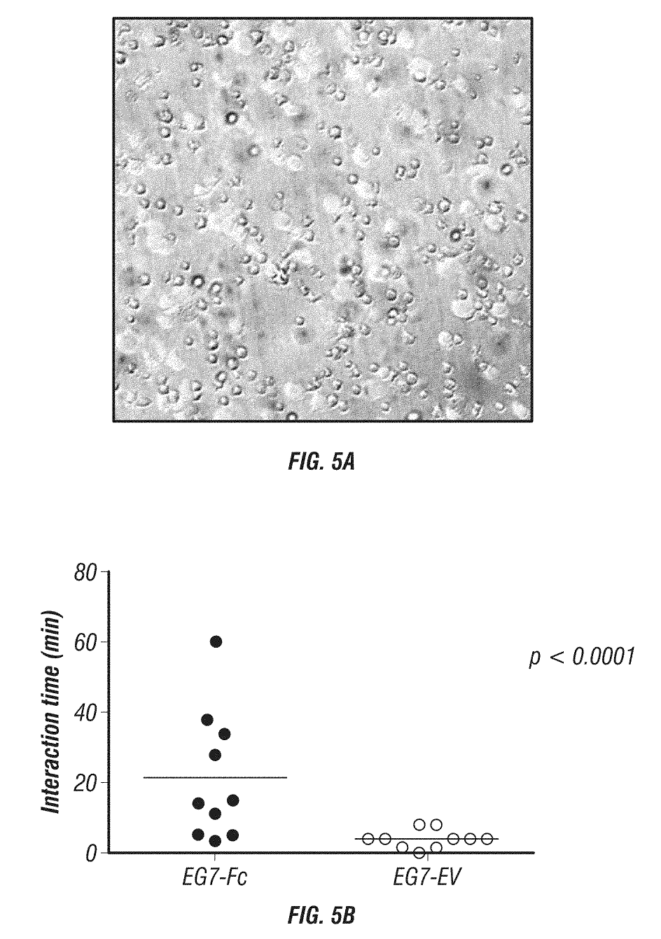

The inventors hypothesized that IgG1-Fc expression on tumor cells would prolong interaction time with DCs and thereby enhance antigen uptake. To test this possibility, the inventors used live cell imaging to observe the interactions between these two cell types in a 4 hour culture. As the tumor cell lines expressed GFP, they were easily differentiated from BMDCs using fluorescence and bright field channels. Tumor/DC interaction events were defined as contacts between the two cell types that were initiated during the 4 hour culture and ceased during the 4 hour culture. The average duration of these interactions was found to be nearly 10 fold longer with EG7-Fc compared to EG7-EV (FIGS. 5A-B). These extended interaction times could potentially lead to enhanced uptake of IgG1-Fc expressing tumor cells by DCs, and result in increased Class I presentation and CD8 priming.

IgG1-Fc Tumors Exhibit Decreased Growth In Vivo and Stimulate Increased Anti-Tumor CD8 T Cell Responses.

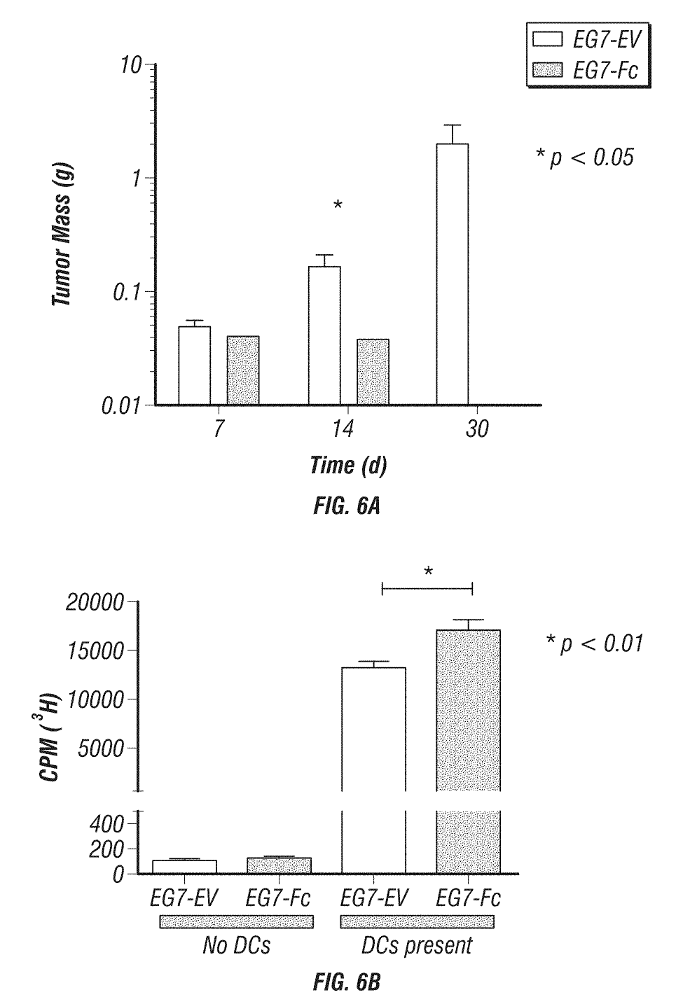

Having observed that tumors expressing IgG1-Fc were able to enhance cross-priming of CD8 T cells in vitro, the inventors wanted to explore the growth and survival of these tumors in vivo. They challenged 15 mice with 500,000 live EG7-Fc or EG7-EV tumor cells injected subcutaneously in the flank. They then euthanized animals on days 7, 14 and 30 (n=5 mice for each time point) and resected all visible tumors. By day 14, the average weight of EG7-Fc tumors was significantly lower than the average weight of EG7-EV tumors (p<0.05 Unpaired T-test). By day 30, no visible tumors were apparent in mice challenged with EG7-Fc tumors, while all control tumors formed large subcutaneous masses (FIG. 6A). To examine the immune response to these tumors, cells collected from draining lymph nodes from tumor bearing mice on day 7 were incubated with purified BMDCs that had been fed tumor cells for 12-16 hours prior to incubation with T cells. CD8 T cells from the draining lymph nodes from EG7-Fc tumor-bearing mice showed higher proliferative responses compared to those from EG7-EV tumor bearing mice (FIG. 6B).

Vaccination with Inactivated IgG1-Fc Tumors Protects Against Subsequent Challenge with Tumor.

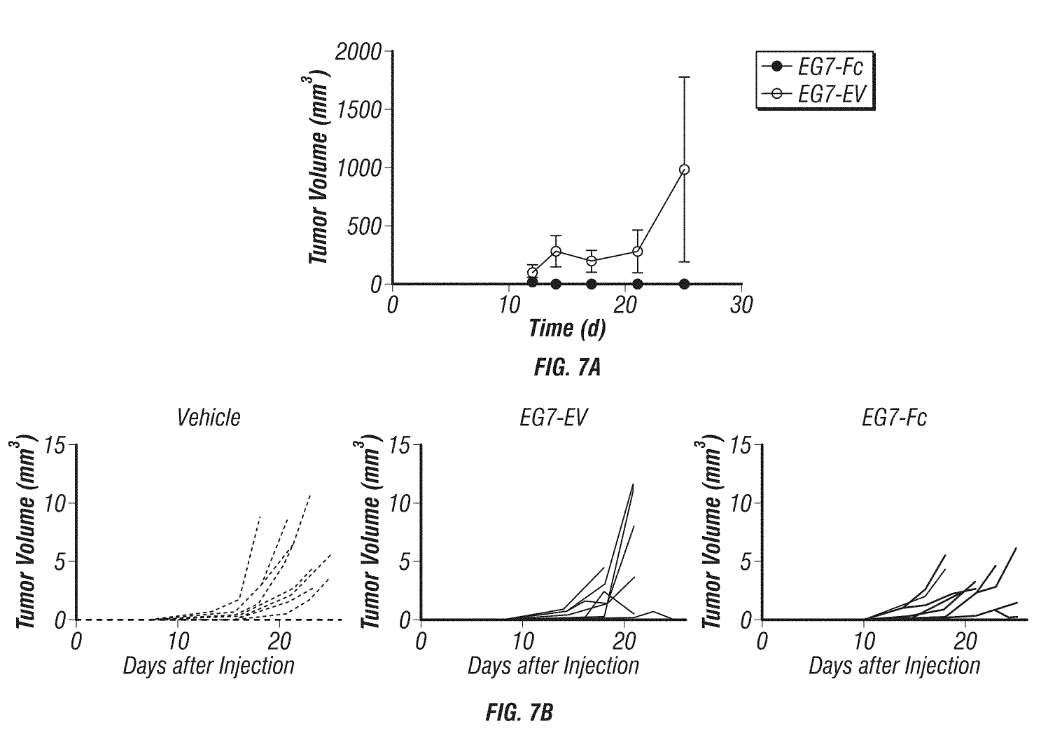

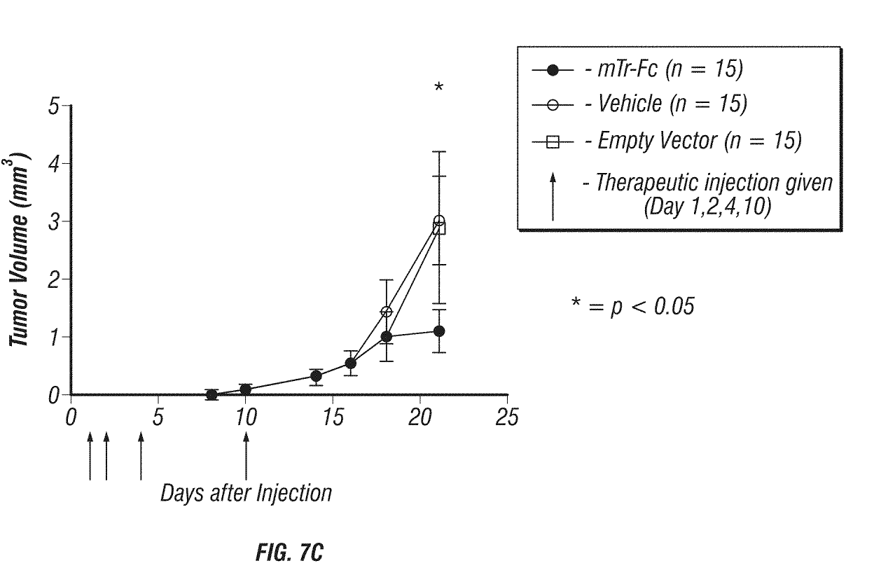

The use of ovalbumin-expressing tumors in the above-described studies allowed us to precisely determine the effects of IgG1-Fc on antigen presentation. The potential power of this approach, however, is that it can effectively induce anti-tumor responses without prior knowledge of tumor-specific antigens. Therefore, in vivo studies using unmanipulated tumors are essential to determine its potential therapeutic utility. To understand if IgG1-Fc expressing tumors induce a memory CD8 response to tumor-specific or tumor-associated antigens in vivo, the inventors tested if treatment of mice with EG7-Fc tumor cells would protect the mice against development of a tumor when challenged with unmanipulated tumor cells (EG7). To ensure that the EG7-Fc and EG7-EV cells used for vaccination would not form primary tumors in vivo, the inventors treated these cells with mitomycin C, a chemotherapeutic agent that is toxic to tumor cell lines, prior to immunization. They established that mitomycin C treatment was sufficient to completely abolish replication as measured by ³H-thymidine incorporation (data not shown). The inventors treated mice with 5×10⁵ mitomycin C inactivated tumor cells (n=5 each group) as a primary vaccine. Twelve days later, mice were challenged with 5×10⁵ EG7 cells in the contra-lateral flank and followed tumor growth by measuring tumor size on days 12, 14, 17, 21 and 25. Mice immunized with mitomycin C treated EG7-Fc expressing cells were less likely to develop measurable tumors than mice immunized with EG7-EV tumor cells (FIG. 7A). These data suggest that IgG1 Fc expressing tumor cells can induce an adaptive immune response that is long-lasting and can prevent growth of an unmanipulated parent tumor cell at a later time point. Taken together, these data suggest that this may be a highly effective approach for prophylactic cancer vaccination.

IgG1-Fc Tumors are Effective as Therapeutic Whole Cell Tumor Vaccines.

To evaluate the efficacy of EG7-Fc as a therapeutic approach to treating established tumors, the inventors implanted unmanipulated EG7 cells on day 0 and subsequently injected mice with live EG7-Fc or EG7-EV tumor cells in the contra lateral flank on days 1, 2, 4 and 10. This strategy was designed to approximate vaccination following surgical removal of a primary tumor where a small number of replicating cells can serve as a source of relapse. The sizes of the primary tumors were measured on day 7, 10, 14, 16, 18 and 21 in a blinded fashion. Mice treated with EG7-Fc had significantly smaller primary tumors by day 18 (vehicle) and day 21 (Empty Vector) (n=15 mice each group) (FIGS. 7B-C). In addition, injection of Fc-bearing tumors did not lead to the development of secondary tumors, while mice that received non-Fc bearing tumor cells developed several secondary tumors, consistent with the inventors' earlier data. These data argue that the immune response generated by Fc-expressing tumors has the ability to halt or reverse the growth of a previously established parental tumor.

### Example 3—Discussion

The lack of effective presentation of tumor specific or tumor-associated antigens to the immune system continues to be a major obstacle in tumor immunotherapy. Known barriers to effective antitumor immune responses include the immunosuppressive tumor microenvironment, lack of cross-presentation of tumor antigen, and blunted effector responses (Pardol, 2012). The inventors present here an approach that targets genetically modified tumors to DCs through transgenic expression of the Fc fragment of IgG1 on the tumor cell surface. Consequently, DC uptake of IgG1-Fc bearing tumors leads to cross-priming of CD8 T cells. In vivo, this approach proved beneficial in promoting shrinkage of pre-existing tumors in mice that were therapeutically “vaccinated” with IgG1-Fc bearing tumor cells. This approach circumvents the requirement for prior knowledge of the tumor antigens that can lead to effective CD8 T cell activation and could have therapeutic potential for a broad spectrum of human cancers.

Polymorphisms in Fc gamma receptors have been associated with an improved clinical response to targeted tumor-associated monoclonal antibodies, suggesting that interactions between the Fc portion of the antibodies and their receptors are important mediators of antitumor responses (Ferris et al., 2010). This, coupled with the evidence that Fc-FcR interactions are important for the uptake, internalization and presentation of antigen to CTLs (den Haan and Bevan, 2002; Dhodapkar et al., 2002) makes enhancing the cross-presentation of tumor antigen an attractive strategy for improving anti-tumor immunity. Furthermore, it is rational to believe that enhanced cross-presentation may be able to diminish tolerance to tumor antigens as one study found that antibody-mediated cross-presentation of antigen can break T cell tolerance in a mouse model of type 1 diabetes (Harbers et al., 2007). The inventors' observation that expression of IgG1 Fc on the surface of tumor cells was able to enhance cross-presentation of tumor-specific antigens and produce measurable clinical efficacy in tumor clearance suggest that this is an immunotherapeutic strategy that is functionally achievable in vivo.

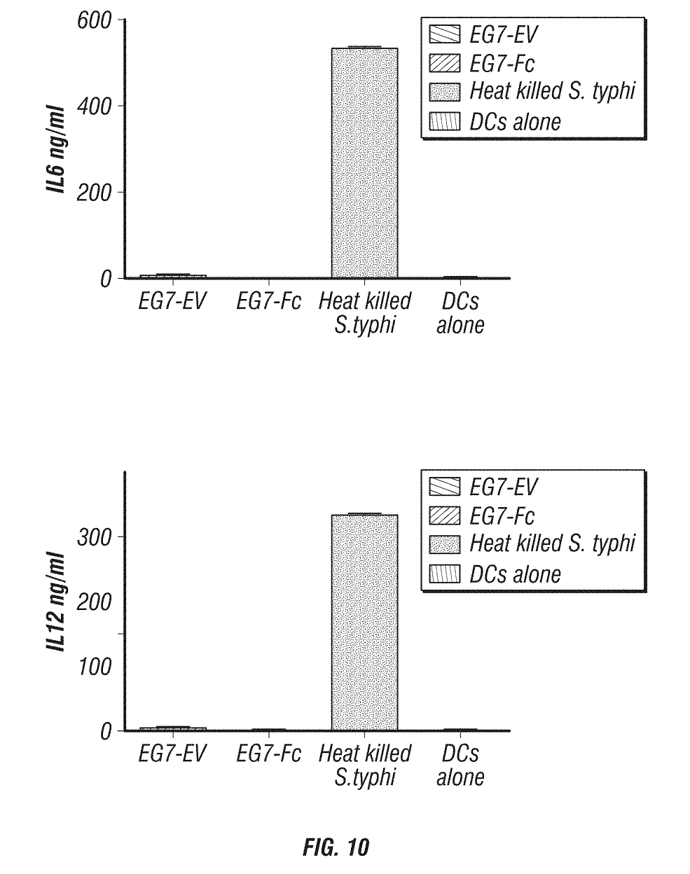

In contrast to a previous study using immune complexes to deliver antigen for cross-presentation (Regnault et al., 1999), these findings suggest that Fc engagement and enhanced cross-priming is not associated with overt DC maturation, as measured by upregulation of CD40 and CD86 (FIGS. 9A-B) and secretion of pro-inflammatory cytokines such as IL-6 and IL-12 (FIGS. 10A-B). One possible explanation for this difference is that cross-presentation induced by immune complexes is qualitatively different from cross-presentation induced by cell-associated Fc. These data showing that cross-priming by BMDCs can be further augmented by ligation of TLR3 suggest that DCs simultaneously activated via PRRs may induce quantitatively higher responses. Further experiments are needed to determine whether combining cell-associated Fc engagement with TLR ligation can influence the quality of CD8 T cell responses against tumor antigens in vivo.

Dendritic cells have been shown to capture antigen from virally-infected cells and cross-present them to CTLs in a process called nibbling (Harshyne et al., 2001). These data argue that prolonged dendritic cell-tumor cell interactions result in enhanced cross-presentation and cross-priming. Further experiments are needed to determine whether the prolonged interactions between DCs and FcR-expressing tumor cells result in a quantitative difference in the number of MHC Class I molecules loaded with antigen, or whether some other mechanism may be responsible for the enhanced cross-priming of CTLs the inventors observed. Importantly, targeting of the tumor cells via Fc receptors enhanced cross-presentation by both bone marrow derived myeloid DCs and a heterogeneous population of DCs obtained after FLT3 ligand administration in vivo, suggesting the utility of this approach in humans may not be limited to targeting of Fc-expressing tumors to specific DC subsets (Nierkens et al. 2013). However, it remains to be seen whether DCs are the primary cells that acquire and cross-present tumor antigens in vivo following Fc-expressing tumor cell vaccination. Notably, it has been suggested previously that macrophages are the primary cell type that cross-presents tumor antigen and primes CD8 T cells in vivo (Asano et al., 2011; Tseng et al., 2013).

A surprising outcome of these experiments is that Fc receptor-mediated targeting of tumor cells to DCs in vitro does not appear to enhance MHC Class II-mediated antigen presentation. Similar findings have been reported by a recent study where enhanced cancer cell phagocytosis by macrophages using anti-CD47 antibody led to increased priming of CD8 T cells but not of CD4 T cells (Tseng et al., 2013). One possible explanation for this observation is that CD8 T cells have much lower requirements for costimulation compared to CD4 T cells (Pardigon et al., 1998; Shahinian et al., 1993). Thus, these results may be explained by the fact that the inventors see no upregulation of costimulatory molecules by DCs incubated with EG7-Fc cells; however, stimulation of DCs using poly I:C also failed to enhance CD4 T cell activation, while CD8 responses were significantly increased. These data imply that antigenic cargo is handled very differently when targeted via the Fc receptors and suggest the possibility that cross-presentation of antigens on MHC Class I is a preferred pathway when DCs take up cells expressing the Fc portion of IgG1. This could have additional benefits in vivo since treatment with Fc-bearing tumors presumably would not induce unwanted CD4 T cell responses against self-antigens that may result in inflammation or auto-immunity. Nonetheless, the modest CD4 T cell responses that are generated in response to Fc-bearing tumors are clearly sufficient to provide the necessary help to CD8 T cells for generation of memory, as both prophylactic and therapeutic approaches using EG7-Fc are highly effective (Janssen et al., 2003). It is also possible that CD4 help in the context of cross-presentation is not necessary for CD8 responses, which could make this approach very attractive for generating long-lasting anti-tumor CD8 responses. Since this work involves use of ovalbumin expressing tumor cells, further investigation is needed to determine if this approach will work to induce CD8 responses against native tumor-derived antigens.

A number of approaches have been taken to improve tumor immunogenicity via the genetic modification of tumors. In a manner similar to this approach, one group induced ectopic surface expression of a chimeric protein of IgG2a and CD98 on murine melanoma cells (Riddle et al., 2005). IgG2a is thought to be more effective at promoting ADCC than IgG1. Notably, these investigators did not observe a significant survival benefit of ectopic IgG2a expression in vivo.

Remarkably, recently established immunotherapies using antibodies that block endogenous immunoregulatory pathways have resulted in cures of tumors that were resistant to conventional treatments (Pardol, 2012). Not all patients, however, respond to these therapies, and their immune-mediated side effects can be debilitating. Therefore, there are three major obstacles to effective tumor immunotherapy. First, anti-tumor immunity must be generated. Second, the immunosuppressive conditions in the tumor stroma must be alleviated. Third, the immunopathological side effects of broader therapeutics, such as CTLA4-targeted agents, must be mitigated. The approach described here addresses the first obstacle and suggests the third obstacle can be minimized. Specifically, vaccination of patients with their own tumors that have been modified to express the Fc region of IgG1 on their surface may initiate adaptive immune responses to their primary tumor and have therapeutic value as a tumor vaccine. Further, relapse in patients harboring residual minimal disease after appropriate conventional therapies may be prevented by the presence of circulating memory anti-tumor lymphocytes. These experiments thus provide a foundation for the development of an effective whole-cell therapeutic cancer vaccine strategy. Further work will determine if this strategy, alone or in combination with other potentially cooperating therapeutics, represents a promising therapeutic avenue towards improving outcomes for cancer patients.

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods, and in the steps or in the sequence of steps of the methods described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### VI. REFERENCES

The following references, to the extent that they provide exemplary procedural or other details supplementary to those set forth herein, are specifically incorporated herein by reference.
Wagle N, Emery C, Berger M F, Davis M J, Sawyer A, Pochanard P, et al. Dissecting therapeutic resistance to RAF inhibition in melanoma by tumor genomic profiling. J Clin Oncol 2011; 29:3085-96.
Rosenberg S A, Restifo N P, Yang J C, Morgan R A, Dudley M E. Adoptive cell transfer: a clinical path to effective cancer immunotherapy. Nat Rev Cancer 2008; 8:299-308. Palucka K, Banchereau J. Cancer immunotherapy via dendritic cells. Nat Rev Cancer 2012; 12:265-77.
Pardoll D M. The blockade of immune checkpoints in cancer immunotherapy. Nat Rev Cancer 2012; 12:252-64.
Iwasaki A, Medzhitov R. Regulation of adaptive immunity by the innate immune system. Science (New York, N.Y.) 2010; 327:291-5.
Kantoff P W, Higano C S, Shore N D, Berger E R, Small E J, Penson D F, et al. Sipuleucel-T immunotherapy for castration-resistant prostate cancer. N Engl J Med 2010; 363:411-22.
Joffre O P, Segura E, Savina A, Amigorena S. Cross-presentation by dendritic cells. Nat Rev Immunol 2012; 12:557-69.
Topalian S L, Weiner G J, Pardoll D M. Cancer immunotherapy comes of age. J Clin Oncol 2011; 29:4828-36.
Schuler G, Schuler-Thurner B, Steinman R M. The use of dendritic cells in cancer immunotherapy. Curr Opin Immunol 2003; 15:138-47.
Bousso P, Robey E. Dynamics of CD8+ T cell priming by dendritic cells in intact lymph nodes. Nat Immunol 2003; 4:579-85.
Janeway C A, Jr. Approaching the asymptote? Evolution and revolution in immunology. Cold Spring Harb Symp Quant Biol 1989; 54 Pt 1:1-13.
Flinsenberg T W, Compeer E B, Koning D, Klein M, Amelung F J, van Baarle D, et al. Fcgamma receptor antigen targeting potentiates cross-presentation by human blood and lymphoid tissue BDCA-3+ dendritic cells. Blood 2012; 120:5163-72.
Cruz L J, Rueda F, Cordobilla B, Simon L, Hosta L, Albericio F, et al. Targeting nanosystems to human DCs via Fc receptor as an effective strategy to deliver antigen for immunotherapy. Mol Pharm 2011; 8:104-16.
Steinman R M. Decisions about dendritic cells: past, present, and future. Annu Rev Immunol 2012; 30:1-22.
Nimmerjahn F, Ravetch J V. Fcgamma receptors as regulators of immune responses. Nat Rev Immunol 2008; 8:34-47.
Ravetch J V, Bolland S. IgG Fc receptors. Annu Rev Immunol 2001; 19:275-90.
Nimmerjahn F, Ravetch J V. Antibodies, Fc receptors and cancer. Curr Opin Immunol 2007; 19:239-45.
Desai D D, Harbers S O, Flores M, Colonna L, Downie M P, Bergtold A, et al. Fc gamma receptor IIB on dendritic cells enforces peripheral tolerance by inhibiting effector T cell responses. J Immunol 2007; 178:6217-26.
Boross P, Leusen J H. Mechanisms of action of CD20 antibodies. Am J Cancer Res 2012; 2:676-90.
Pokrass M J, Liu M F, Lindorfer M A, Taylor R P. Activation of complement by monoclonal antibodies that target cell-associated beta-microglobulin: Implications for cancer immunotherapy. Mol Immunol 2013; 56:549-60.
Boross P, Lohse S, Nederend M, Jansen J H, van Tetering G, Dechant M, et al. IgA EGFR antibodies mediate tumour killing in vivo. EMBO Mol Med 2013; 5:1213-26.
Scott A M, Wolchok J D, Old L J. Antibody therapy of cancer. Nat Rev Cancer 2012; 12:278-87.
Signorino E, Brusa D, Granata R, Malavasi F, Ferrone S, Matera L. Contribution of dendritic cells&apos; FcgammaRI and FcgammaRIII to cross-presentation of tumor cells opsonized with the anti-MHC class I monoclonal antibodies. Cancer biology &amp; therapy 2007; 6:1932-7.
Dhodapkar K M, Krasovsky J, Williamson B, Dhodapkar M V. Antitumor monoclonal antibodies enhance cross-presentation ofcCellular antigens and the generation of myeloma-specific killer T cells by dendritic cells. J Exp Med 2002; 195:125-33.
Weiner L M, Dhodapkar M V, Ferrone S. Monoclonal antibodies for cancer immunotherapy. Lancet 2009; 373:1033-40.
Rafiq K, Bergtold A, Clynes R. Immune complex-mediated antigen presentation induces tumor immunity. J Clin Invest 2002; 110:71-9.
Harbers S O, Crocker A, Catalano G, D'Agati V, Jung S, Desai D D, et al. Antibody-enhanced cross-presentation of self antigen breaks T cell tolerance. J Clin Invest 2007; 117:1361-9.
Getahun A, Dahlstrom J, Wernersson S, Heyman B. IgG2a-mediated enhancement of antibody and T cell responses and its relation to inhibitory and activating Fc gamma receptors. J Immunol 2004; 172:5269-76.
Regnault A, Lankar D, Lacabanne V, Rodriguez A, Thery C, Rescigno M, et al. Fcgamma receptor-mediated induction of dendritic cell maturation and major histocompatibility complex class I-restricted antigen presentation after immune complex internalization. J Exp Med 1999; 189:371-80.
Amigorena S. Fc gamma receptors and cross-presentation in dendritic cells. J Exp Med 2002; 195:F1-3.
den Haan J M, Bevan M J. Constitutive versus activation-dependent cross-presentation of immune complexes by CD8(+) and CD8(−) dendritic cells in vivo. J Exp Med 2002; 196:817-27.
Stabila P F, Wong S C, Kaplan F A, Tao W. Cell surface expression of a human IgG Fc chimera activates macrophages through Fc receptors. Nat Biotechnol 1998; 16:1357-60.
Takashima Y, Tsukamoto M, Ota H, Matsumoto Y, Hayashi Y, Otsuka H. Immunization with pseudorabies virus harboring Fc domain of IgG makes a contribution to protection of mice from lethal challenge. Vaccine 2005; 23:3775-82.
Moore M W, Carbone F R, Bevan M J. Introduction of soluble protein into the class I pathway of antigen processing and presentation. Cell 1988; 54:777-85.
Mayordomo J I, Zorina T, Storkus W J, Zitvogel L, Celluzzi C, Falo L D, et al. Bone marrow-derived dendritic cells pulsed with synthetic tumour peptides elicit protective and therapeutic antitumour immunity. Nat Med 1995; 1:1297-302.
Mach N, Gillessen S, Wilson S B, Sheehan C, Mihm M, Dranoff G. Differences in dendritic cells stimulated in vivo by tumors engineered to secrete granulocyte-macrophage colony-stimulating factor or Flt3-ligand. Cancer Res 2000; 60:3239-46.
Clarke S R, Barnden M, Kurts C, Carbone F R, Miller J F, Heath W R. Characterization of the ovalbumin-specific TCR transgenic line OT-I: MHC elements for positive and negative selection. Immunol Cell Biol 2000; 78:110-7.
Barnden M J, Allison J, Heath W R, Carbone F R. Defective TCR expression in transgenic mice constructed using cDNA-based alpha- and beta-chain genes under the control of heterologous regulatory elements. Immunol Cell Biol 1998; 76:34-40.
Longhi M P, Trumpfheller C, Idoyaga J, Caskey M, Matos I, Kluger C, et al. Dendritic cells require a systemic type I interferon response to mature and induce CD4+Th1 immunity with poly IC as adjuvant. J Exp Med 2009; 206:1589-602.
Ferris R L, Jaffee E M, Ferrone S. Tumor antigen-targeted, monoclonal antibody-based immunotherapy: clinical response, cellular immunity, and immunoescape. J Clin Oncol 2010; 28:4390-9.
Dhodapkar K M, Krasovsky J, Williamson B, Dhodapkar M V. Antitumor monoclonal antibodies enhance cross-presentation of Cellular antigens and the generation of myeloma-specific killer T cells by dendritic cells. J Exp Med 2002; 195:125-33.
Harbers S O, Harbers S O, Crocker A, Crocker A, Catalano G, Catalano G, et al. Antibody-enhanced cross-presentation of self antigen breaks T cell tolerance. Journal of Clinical Investigation 2007; 117:1361-9.
Regnault A, Lankar D, Lacabanne V, Rodriguez A, Théry C, Rescigno M, et al. Fcgamma receptor-mediated induction of dendritic cell maturation and major histocompatibility complex class I-restricted antigen presentation after immune complex internalization. J Exp Med 1999; 189:371-80.
Harshyne L A, Watkins S C, Gambotto A, Barratt-Boyes S M. Dendritic cells acquire antigens from live cells for cross-presentation to CTL. J Immunol 2001; 166:3717-23.
Nierkens S, Tel J, Janssen E, Adema G J. Antigen cross-presentation by dendritic cell subsets: one general or all sergeants? Trends Immunol 2013; 34:361-70.
Asano K, Nabeyama A, Miyake Y, Qiu C H, Kurita A, Tomura M, et al. CD169-positive macrophages dominate antitumor immunity by crosspresenting dead cell-associated antigens. Immunity 2011; 34:85-95.
Tseng D, Volkmer J P, Willingham S B, Contreras-Trujillo H, Fathman J W, Fernhoff N B, et al. Anti-CD47 antibody-mediated phagocytosis of cancer by macrophages primes an effective antitumor T-cell response. Proc Natl Acad Sci USA 2013; 110:11103-8.
Pardigon N, Bercovici N, Calbo S, Santos-Lima E C, Liblau R, Kourilsky P, et al. Role of co-stimulation in CD8+ T cell activation. Int Immunol 1998; 10:619-30.
Shahinian A, Pfeffer K, Lee K P, Kundig T M, Kishihara K, Wakeham A, et al. Differential T cell costimulatory requirements in CD28-deficient mice. Science 1993; 261:609-12.
Janssen E M, Lemmens E E, Wolfe T, Christen U, von Herrath M G, Schoenberger S P. CD4+ T cells are required for secondary expansion and memory in CD8+ T lymphocytes. Nature 2003; 421:852-6.
Riddle D S, Sanz L, Chong H, Thompson J, Vile R G. Tumor cell surface display of immunoglobulin heavy chain Fc by gene transfer as a means to mimic antibody therapy. Hum Gene Ther 2005; 16:830-44.
Pasare C, Medzhitov R. Toll pathway-dependent blockade of CD4+CD25+ T cell-mediated suppression by dendritic cells. Science 2003; 299:1033-6.
Pasare C, Medzhitov R. Toll-dependent control mechanisms of CD4 T cell activation. Immunity 2004; 21:733-41.
Euhus D M, Hudd C, LaRegina M C, Johnson F E. Tumor measurement in the nude mouse. J Surg Oncol 1986; 31:229-34.
U.S. Pat. No. 4,683,202
U.S. Pat. No. 4,684,611
U.S. Pat. No. 4,879,236
U.S. Pat. No. 4,952,500
U.S. Pat. No. 5,021,236
U.S. Pat. No. 5,217,879
U.S. Pat. No. 5,302,523
U.S. Pat. No. 5,322,783
U.S. Pat. No. 5,384,253
U.S. Pat. No. 5,464,765
U.S. Pat. No. 5,506,138
U.S. Pat. No. 5,538,877
U.S. Pat. No. 5,538,880
U.S. Pat. No. 5,550,318
U.S. Pat. No. 5,563,055
U.S. Pat. No. 5,580,859
U.S. Pat. No. 5,589,466
U.S. Pat. No. 5,610,042
U.S. Pat. No. 5,656,610
U.S. Pat. No. 5,670,488
U.S. Pat. No. 5,702,932
U.S. Pat. No. 5,736,524
U.S. Pat. No. 5,739,018
U.S. Pat. No. 5,780,448
U.S. Pat. No. 5,789,215
U.S. Pat. No. 5,824,544
U.S. Pat. No. 5,830,725
U.S. Pat. No. 5,849,304
U.S. Pat. No. 5,851,826
U.S. Pat. No. 5,858,744
U.S. Pat. No. 5,871,983
U.S. Pat. No. 5,871,986
U.S. Pat. No. 5,879,934
U.S. Pat. No. 5,888,502
U.S. Pat. No. 5,928,906
U.S. Pat. No. 5,932,210
U.S. Pat. No. 5,945,100
U.S. Pat. No. 5,955,331
U.S. Pat. No. 5,981,274
U.S. Pat. No. 5,994,136
U.S. Pat. No. 5,994,624
U.S. Pat. No. 6,013,516
“Antibodies: A Laboratory Manual,” Cold Spring Harbor Press, Cold Spring Harbor, N.Y., 1988.
Almendro et al., J. Immunol., 157(12):5411-5421, 1996.
Amado and Chen, Science, 285(5428):674-676, 1999.
Angel et al., Cell, 49:729, 1987a.
Angel et al., Cell, 49:729, 1987b.
Armentano et al., Proc. Natl. Acad. Sci. USA, 87(16):6141-6145, 1990.
Ausubel et al., Current Protocols in Molecular Biology, Greene Publishing Associates and Wiley Interscience, N.Y., 1994.
Banerji et al., Cell, 27:299, 1981.
Banerji et al., Cell, 33(3):729-740, 1983.
Bates, Mol. Biotechnol., 2(2):135-145, 1994.
Batra et al., Am. J. Respir. Cell Mol. Biol., 21(2):238-245, 1999.
Battraw and Hall, Theor. App. Genet., 82(2):161-168, 1991.
Berkhout et al., Cell, 59:273-282, 1989.
Bett et al., J. Virololgy, 67(10):5911-5921, 1993.
Bhattacharjee et al., J. Plant Bioch. Biotech., 6(2):69-73. 1997.
Bilbao et al., FASEB J., 11(8):624-634, 1997.
Blackwell et al., Arch. Otolaryngol Head Neck Surg., 125(8):856-863, 1999.
Blanar et al., EMBO J., 8:1139, 1989.
Blomer et al., J. Virol., 71(9):6641-6649, 1997.
Bodine and Ley, EMBO J., 6:2997, 1987.
Boshart et al., Cell, 41:521, 1985.
Bosze et al., EMBO J., 5(7):1615-1623, 1986.
Braddock et al., Cell, 58:269, 1989.
Bulla and Siddiqui, J. Virol., 62:1437, 1986.
Campbell and Villarreal, Mol. Cell. Biol., 8:1993, 1988.
Campbell, In: Monoclonal Antibody Technology, Laboratory Techniques in Biochemistry and Molecular Biology, Vol. 13, Burden and Von Knippenberg, Eds. pp. 75-83, Amsterdam, Elsevier, 1984.
Campere and Tilghman, Genes and Dev., 3:537, 1989.
Campo et al., Nature, 303:77, 1983.
Caplen et al., Gene Ther., 6(3):454-459, 1999.
Carbonelli et al., FEMS Microbiol. Lett., 177(1):75-82, 1999.
Case et al., Proc. Natl. Acad. Sci. USA, 96(6):2988-2993, 1999.
Celander and Haseltine, J. Virology, 61:269, 1987.
Celander et al., J. Virology, 62:1314, 1988.
Chandler et al., Proc. Natl. Acad. Sci. USA, 94(8):3596-601, 1997.
Chang et al., Mol. Cell. Biol., 9:2153, 1989.
Chatterjee et al., Proc. Natl. Acad. Sci. USA, 86:9114, 1989.
Chen and Okayama, Mol. Cell Biol., 7(8):2745-2752, 1987.
Chillon et al., J. Virol., 73(3):2537-2540, 1999.
Christou et al., Proc. Natl. Acad. Sci. USA, 84(12):3962-3966, 1987.
Clay et al., J. Immunol., 162:1749, 1999.
Cocea, Biotechniques, 23(5):814-816, 1997.
Coffey et al., Science, 282(5392):1332-1334, 1998.
Cohen et al., J. Cell. Physiol., 5:75, 1987.
Costa et al., Mol. Cell. Biol., 8:81-90, 1988.
Cripe et al., EMBO J., 6:3745, 1987.
Culotta and Hamer, Mol. Cell. Biol., 9:1376-1380, 1989.
D'Halluin et al., Plant Cell, 4(12):1495-1505, 1992.
Dandolo et al., J. Virology, 47:55-64, 1983.
DeLuca et al., J. Virol., 56(2):558-570, 1985.
Derby et al., Hear Res, 134(1-2):1-8, 1999.
Deschamps et al., Science, 230:1174-1177, 1985.
Dorai et al., Int. J. Cancer, 82(6):846-52, 1999.
Edbrooke et al., Mol. Cell. Biol., 9:1908-1916, 1989.
Edlund et al., Science, 230:912-916, 1985.
Engel and Kohn, Front Biosci, 4:e26-33, 1999. EPO 0273085
Fechheimer et al., Proc Natl. Acad. Sci. USA, 84:8463-8467, 1987.
Feldman et al., Cardiovasc. Res., 32(2):194-207, 1996.
Feldman et al., Semin. Interv. Cardiol., 1(3):203-208, 1996.
Feng and Holland, Nature, 334:6178, 1988.
Feng et al., Nat. Biotechnol., 15(9):866-870, 1997.
Firak and Subramanian, Mol. Cell. Biol., 6:3667, 1986.
Fisher et al., Hum. Gene Ther., 7(17):2079-2087, 1996.
Foecking and Hofstetter, Gene, 45(1):101-105, 1986.
Fraley et al., Proc. Natl. Acad. Sci. USA, 76:3348-3352, 1979.
Fujita et al., Cell, 49:357, 1987.
Fujiwara and Tanaka, Nippon Geka Gakkai Zasshi, 99(7):463-468, 1998.
Garoff and Li, Curr. Opin. Biotechnol., 9(5):464-469, 1998.
Garrido et al., J. Neurovirol., 5(3):280-288, 1999.
Ghosh and Bachhawat, In: Liver Diseases, Targeted Diagnosis and Therapy Using Specific Receptors and Ligands, Wu et al. (Eds.), Marcel Dekker, NY, 87-104, 1991.
Gloss et al., EMBO J., 6:3735, 1987.
Gnant et al., Cancer Res., 59(14):3396-403, 1999.
Gnant et al., J. Natl. Cancer Inst., 91(20):1744-1750, 1999.
Godbout et al., Mol. Cell. Biol., 8:1169, 1988.
Goodboum et al., Cell, 45:601, 1986.
Gopal, Mol. Cell Biol., 5:1188-1190, 1985.
Graham and Prevec, Mol Biotechnol, 3(3):207-220, 1995.
Graham and Van Der Eb, Virology, 52:456-467, 1973.
Greene et al., Immunology Today, 10:272, 1989.
Grosschedl and Baltimore, Cell, 41:885, 1985.
Haecker et al., Hum. Gene Ther., 7(15):1907-1914, 1996.
Harland and Weintraub, J. Cell Biol., 101(3):1094-1099, 1985.
Haslinger and Karin, Proc. Natl. Acad. Sci. USA, 82:8572, 1985.
Hauber and Cullen, J. Virology, 62:673, 1988.
Hayashi et al., Neurosci. Lett., 267(1):37-40, 1999.
He et al., Plant Cell Reports, 14 (2-3):192-196, 1994.
Hen et al., Nature, 321:249, 1986.
Hensel et al., Lymphokine Res., 8:347, 1989.
Hermens and Verhaagen, Prog. Neurobiol., 55(4):399-432, 1998.
Herr and Clarke, Cell, 45:461, 1986.
Hirochika et al., J. Virol., 61:2599, 1987.
Holbrook et al., Virology, 157:211, 1987.
Holzer et al. Virology, 253(1):107-114, 1999.
Horlick and Benfield, Mol. Cell. Biol., 9:2396, 1989.
Hou and Lin, Plant Physiology, 111:166, 1996.
Howard et al., Ann. NY Acad. Sci., 880:352-365, 1999.
Huang et al., Cell, 27:245, 1981.
Huard et al., Neuromuscul Disord, 7(5):299-313, 1997.
Hug et al., Mol. Cell. Biol., 8:3065-3079, 1988.
Hwang et al., Mol. Cell. Biol., 10:585, 1990.
Imagawa et al., Cell, 51:251, 1987.
Imai et al., Nephrologie, 19(7):397-402, 1998.
Imbra and Karin, Nature, 323:555, 1986.
Imler et al., Mol. Cell. Biol., 7:2558, 1987.
Imperiale and Nevins, Mol. Cell. Biol., 4:875, 1984.
Irie et al., Antisense Nucleic Acid Drug Dev., 9(4):341-349, 1999.
Jakobovits et al., Mol. Cell. Biol., 8:2555, 1988.
Jameel and Siddiqui, Mol. Cell. Biol., 6:710, 1986.
Jaynes et al., Mol. Cell. Biol., 8:62, 1988.
Johnson et al., Mol. Cell. Biol., 9(8):3393-3399, 1989.
Johnston et al., J. Virol., 73(6):4991-5000, 1999.
Kadesch and Berg, Mol. Cell. Biol., 6:2593, 1986.
Kaeppler et al., Plant Cell Rep., 8:415-418, 1990.
Kaneda et al., Science, 243:375-378, 1989.
Karin et al., Mol. Cell. Biol., 7:606, 1987.
Katinka et al., Cell, 20:393, 1980.
Kato et al, J. Biol. Chem., 266:3361-3364, 1991.
Kaufman et al., Arch. Ophthalmol., 117(7):925-928, 1999.
Kawamoto et al., Mol. Cell. Biol., 8:267, 1988.
Kay, Haemophilia, 4(4):389-392, 1998.
Kiledjian et al., Mol. Cell. Biol., 8:145, 1988.
Klamut et al., Mol. Cell. Biol., 10:193, 1990.
Klimatcheva et al., Front Biosci, 4:D481-496, 1999.
Koch et al., Mol. Cell. Biol., 9:303, 1989.
Kohut et al., Am. J. Physiol., 275(6Pt1):L1089-1094, 1998.
Kooby et al., FASEB J, 13(11):1325-34, 1999.
Kraus et al. FEBS Lett., 428(3):165-170, 1998.
Kriegler and Botchan, Mol. Cell. Biol., 3:325, 1983.
Kriegler et al., Cell, 38:483, 1984a.
Kriegler et al., In: Cancer Cells 2/Oncogenes and Viral Genes, Van de Woude et al. eds, Cold Spring Harbor, Cold Spring Harbor Laboratory, 1984b.
Krisky et al., Gene Ther, 5(11):1517-1530, 1998a.
Krisky et al., Gene Ther, 5(12):1593-1603, 1998b.
Kuhl et al., Cell, 50:1057, 1987.
Kunz et al., Nucl. Acids Res., 17:1121, 1989.
Lachmann and Efstathiou, Curr. Opin. Mol. Ther., 1(5):622-632, 1999.
Lareyre et al., J. Biol. Chem., 274(12):8282-8290, 1999.
Larsen et al., Proc. Natl. Acad. Sci. USA, 83:8283, 1986.
Laspia et al., Cell, 59:283, 1989.
Latimer et al., Mol. Cell. Biol., 10:760, 1990.
Lazzeri, Methods Mol. Biol., 49:95-106, 1995.
Lee et al., Environ. Mol. Mutagen., 13(1):54-59, 1989.
Lee et al., Nature, 294:228, 1981.
Lee et al., Nucleic Acids Res., 12:4191-206, 1984.
Leibowitz et al., Diabetes, 48(4):745-753, 1999.
Lesch, Biol Psychiatry, 45(3):247-253, 1999.
Levenson et al., Hum. Gene Ther., 9(8):1233-1236, 1998.
Lin et al., Mol. Cell. Biol., 10:850, 1990.
Lundstrom, J Recept Signal Transduct. Res., 19(1-4):673-686, 1999.
Luria et al., EMBO J., 6:3307, 1987.
Lusky and Botchan, Proc. Natl. Acad. Sci. USA, 83:3609, 1986.
Lusky et al., Mol. Cell. Biol., 3:1108, 1983.
Majors and Varmus, Proc. Natl. Acad. Sci. USA, 80:5866, 1983.
Marienfeld et al., Gene Ther., 6(6):1101-1113, 1999.
Mastrangelo et al., Biotechnol. Bioeng., 65(3):298-305, 1999.
McNeall et al., Gene, 76:81, 1989.
Miksicek et al., Cell, 46:203, 1986.
Miller et al., J. Pharmacol. Exp. Ther., 264:11-16, 1993.
Miyatake et al., Gene Ther 6:564-572, 1999.
Moldawer et al., Shock, 12(2):83-101, 1999.
Mordacq and Linzer, Genes and Dev., 3:760, 1989.
Moreau et al., Nucl. Acids Res., 9:6047, 1981.
Moriuchi et al., Cancer Res, 58(24):5731-5737, 1998.
Morrison et al., J. Gen. Virol., 78(Pt 4):873-878, 1997.
Muesing et al., Cell, 48:691, 1987.
Naldini et al., Science, 272(5259):263-267, 1996.
Neumann et al., Proc. Natl. Acad. Sci. USA, 96(16):9345-9350, 1999.
Ng et al., Nuc. Acids Res., 17:601, 1989.
Nicolau and Sene, Biochim. Biophys. Acta, 721:185-190, 1982.
Nicolau et al., Methods Enzymol., 149:157-176, 1987.
Nomoto et al., Gene, 236(2):259-271, 1999.
Omirulleh et al., Plant Mol. Biol., 21(3):415-428, 1993.
Ondek et al., EMBO 1, 6:1017, 1987.
Palmiter et al., Cell, 29:701, 1982.
Parks et al., J. Virol., 71(4):3293-8, 1997.
PCT Appln. WO 92/17598
PCT Appln. WO 94/09699
PCT Appln. WO 95/06128
Pech et al., Mol. Cell. Biol., 9:396, 1989.
Perales et al., Proc. Natl. Acad. Sci. USA, 91:4086-4090, 1994.
Perez-Stable and Constantin, Mol. Cell. Biol., 10:1116, 1990.
Petrof, Eur Respir J, 11(2):492-497, 1998.
Picard and Schaffner, Nature, 307:83, 1984.
Pinkert et al., Genes and Dev., 1:268, 1987.
Ponta et al., Proc. Natl. Acad. Sci. USA, 82:1020, 1985.
Potrykus et al., Mol. Gen. Genet., 199(2):169-177, 1985.
Potter and Haley, Meth. Enzymol., 91, 613-633, 1983.
Potter et al., Proc. Natl. Acad. Sci. USA, 81:7161-7165, 1984.
Queen and Baltimore, Cell, 35:741, 1983.
Quinn et al., Mol. Cell. Biol., 9:4713, 1989.
Rabinovitch et al., Diabetes, 48(6):1223-1229, 1999.
Reddy et al., Virology, 251(2):414-26, 1998.
Redondo et al., Science, 247:1225, 1990.
Reisman and Rotter, Mol. Cell. Biol., 9:3571, 1989.
Remington's Pharmaceutical Sciences, 15th Ed., 33:624-652, 1990.
Resendez Jr. et al., Mol. Cell. Biol., 8:4579, 1988.
Rhodes et al., Methods Mol. Biol., 55:121-131, 1995.
Rippe et al., Mol. Cell. Biol., 9(5):2224-22277, 1989.
Rippe, et al., Mol. Cell Biol., 10:689-695, 1990.
Riffling et al., Nucl. Acids Res., 17:1619, 1989.
Robbins and Ghivizzani, Pharmacol Ther, 80(1):35-47, 1998.
Robbins et al., Trends Biotechnol., 16(1):35-40, 1998.
Sambrook et al., In: Molecular cloning: a laboratory manual, 2^{nd} Ed., Cold Spring Harbor Laboratory Press, Cold Spring Harbor, N.Y., 1989.
Sawai et al. Mol. Genet. Metab., 67(1):36-42, 1999.
Schaffner et al., J. Mol. Biol., 201:81, 1988.
Searle et al., Mol. Cell. Biol., 5:1480, 1985.
Sharp and Marciniak, Cell, 59:229, 1989.
Shaul and Ben-Levy, EMBO J., 6:1913, 1987.
Sherman et al., Mol. Cell. Biol., 9:50, 1989.
Sleigh and Lockett, J EMBO, 4:3831, 1985.
Smith et al., Neuron., 20:1093-1102, 1998.
Spalholz et al., Cell, 42:183, 1985.
Spandau and Lee, J. Virology, 62:427, 1988.
Spandidos and Wilkie, EMBO J., 2:1193, 1983.
Stephens and Hentschel, Biochem. 1, 248:1, 1987.
Stewart et al., Arch. Biochem. Biophys. 365:71-74; 1999.
Stuart et al., Nature, 317:828, 1985.
Sullivan and Peterlin, Mol. Cell. Biol., 7:3315, 1987.
Suzuki et al., Biochem Biophys Res Commun, 252(3):686-90, 1998.
Swartzendruber and Lehman, J. Cell. Physiology, 85:179, 1975.
Takebe et al., Mol. Cell. Biol., 8:466, 1988.
Tavernier et al., Nature, 301:634, 1983.
Taylor and Kingston, Mol. Cell. Biol., 10:165, 1990a.
Taylor and Kingston, Mol. Cell. Biol., 10:176, 1990b.
Taylor et al., J. Biol. Chem., 264:15160, 1989.
Thiesen et al., J. Virology, 62:614, 1988.
Timiryasova et al., Int. J. Oncol., 14(5):845-854, 1999.
Treisman, Cell, 42:889, 1985
Tronche et al., Mol. Biol. Med., 7:173, 1990.
Tronche et al., Mol. Cell. Biol., 9:4759, 1989.
Trudel and Constantini, Genes and Dev., 6:954, 1987.
Tsukada et al., Plant Cell Physiol., 30(4)599-604, 1989.
Tsumaki et al., J. Biol. Chem., 273(36):22861-22864, 1998.
Tur-Kaspa et al., Mol. Cell Biol., 6:716-718, 1986.
Tyndall et al., Nuc. Acids. Res., 9:6231, 1981.
Vanderkwaak et al., Gynecol Oncol, 74(2):227-234, 1999.
Vasseur et al., Proc. Natl. Acad. Sci. USA, 77:1068, 1980.
Wagner et al., Proc. Natl. Acad. Sci. USA 87(9):3410-3414, 1990.
Wang and Calame, Cell, 47:241, 1986.
Wang et al., Infect. Immun., 66:4193-202, 1998.
Weber et al., Cell, 36:983, 1984.
Weihl et al., Neurosurgery, 44(2):239-252, 1999.
White et al. J. Virol., 73(4):2832-2840, 1999.
Wilson, J Clin. Invest., 98(11):2435, 1996.
Winoto and Baltimore, Cell, 59:649, 1989.
Wong et al., Gene, 10:87-94, 1980.
Wu and Wu, Adv. Drug Delivery Rev., 12:159-167, 1993.
Wu and Wu, J. Biol. Chem., 262:4429-4432, 1987.
Wu et al., Biochem. Biophys. Res. Commun., 233(1):221-226, 1997.
Yamada et al., Brain Res., 833(2):302-307, 1999.
Yeung et al., Gene Ther., 6(9):1536-1544, 1999.
Yoon et al., J. Gastrointest. Surg., 3(1):34-48, 1999.
Yutzey et al. Mol. Cell. Biol., 9:1397, 1989.
Zhao-Emonet et al., Biochim. Biophys. Acta, 1442(2-3):109-119, 1998.
Zheng et al., J. Gen. Virol., 80(Pt 7):1735-1742, 1999.
Zhou et al., Nature, 361(6412):543-547, 1993.
Zufferey et al., Nat. Biotechnol., 15(9):871-875, 1997.

1. A method of treating cancer in a subject comprising:
(a) providing a recombinant cancer cell that expresses Ig Fc on its surface; and
(b) administering said recombinant cancer cell to said subject.

2-3. (canceled)

4. The method of claim 1, wherein said cancer is a solid tumor.

5. The method of claim 4, wherein said solid tumor is selected from the group consisting of breast cancer, lung cancer, colon cancer, pancreatic cancer, renal cancer, stomach cancer, liver cancer, bone cancer, neural tissue cancer, melanoma, ovarian cancer, testicular cancer, prostate cancer, cervical cancer, vaginal cancer, and bladder cancer.

6. The method of claim 1, wherein said cancer is a hematologic cancer.

7. The method of claim 6, wherein said hematologic cancer is a leukemia or lymphoma.

8. The method of claim 1, wherein said cancer is recurrent, metastatic and/or multi-drug resistant.

9. The method of claim 1, wherein said recombinant cancer cell is autologous to said subject.

10. The method of claim 1, wherein said recombinant cancer cell is not autologous to said subject.

11. The method of claim 1, wherein said recombinant cancer cell is transformed with an expression construct that expresses said Ig Fc.

12-15. (canceled)

16. The method of claim 1, wherein said Ig Fc molecule is an IgG Fc molecule.

17. The method of claim 16, wherein said IgG Fc molecule is an IgG1 Fc molecule.

18. The method of claim 1, further comprising treating said subject with a second cancer therapy.

19. The method of claim 18, wherein said second cancer therapy is surgery, chemotherapy, radiotherapy, gene therapy, toxin therapy, hormone therapy or an immunotherapy.

20. The method of claim 19, wherein said immunotherapy comprises treating said subject with a TLR3 ligand or a RIG-I ligand.

21. The method of claim 1, further comprising assessing the genotype and/or phenotype of said cancer prior to treatment.

22. The method of claim 1, further comprising:
(i) obtaining, prior to treatment, a cancer cell from said subject; and
(ii) engineering said cancer to produce said recombinant cancer cell.

23. The method of claim 1, further comprising assessing an immune response to said recombinant cancer cell after step (b).

24. The method of claim 23, wherein said immune response is a CD8+ T cell response.

25. The method of claim 1, wherein said method inhibits metastasis, inhibits primary tumor growth, and/or induces primary tumor regression.

26-27. (canceled)

28. The method of claim 1, wherein said method reduces tumor burden or renders an unresectable tumor resectable.

29. (canceled)

30. The method of claim 1, wherein said recombinant cancer cell is engineered to express one or more heterologous tumor antigens.

31-50. (canceled)
